(19) United States
(12) Patent Application Publication
(10) Pub. No.: US 2004/0135017 A1
(43) Pub. Date: Jul. 15, 2004

(54) Mixing, crushing, and pulverizing device, and method of pulverizing substances using the device

(76) Inventor: Sukeyoshi Sekine, Shizuoka (JP)

Correspondence Address: WENDEROTH, LIND & PONACK, L.L.P., 2033 K STREET N. W., SUITE 800, WASHINGTON, DC, 20006-1021, US

(21) Appl. No.: 10/476,892

(22) PCT Filed: Jan. 15, 2002
(86) PCT No.: PCT/JP02/00175

(30) Foreign Application Priority Data
May 7, 2001 (JO) 2001-136229

(51) Int. Cl. B02C 19/00

(52) U.S. Cl. 241/15

(57) ABSTRACT

An apparatus for mixing and/or crushing substances into fine particles is disclosed, wherein the apparatus includes a fluid flow path that is formed by a first structural member having a plurality of cells thereon, each having its front side open, and a second structural member having a plurality of cells thereon, each having its front side open; the first structural member and the second structural member are arranged adjacently to each other such that the open front sides of the cells on one structural member face opposite the open front sides of the corresponding cells on the other structural member, with the cells on one structural member being placed in alternate positions with regard to the corresponding cells on the other structural member, and with each of the cells on one structural member communicating with at least one or more of the cells on the other structural member, wherein when a right-angled isosceles triangle is given and when a line segment connecting between any arbitrary point on the two hypotenuses and any arbitrary point on the perpendicular from the vortex to the base is given, each of the cells has the shape as its basic geometric shape having the area that is equal to one half the area of a right-angled isosceles triangle that is obtained by turning the above line segment clockwise or anticlockwise about any arbitrary point on the hypotenuse as the origin to intersect the line segment with the base, and then by connecting those line segments.

A method for mixing and/or crushing substances into fine particles is also disclosed, wherein the method comprises the steps performed by using the before described apparatus, a fluid, in which substances to be crushed are mixed, are introduced into the inlet of the cylindrical casing under the applied pressure, and subjecting the substances to the mixing and/or crushing process while flowing the said fluid through the fluid flow path.

### BACKGROUND

1. Technical Field

The present invention relates to an apparatus for mixing various substances into a fluid, an apparatus for crushing various substances into fine particles, an apparatus for mixing and crushing various substances into fine particles, and a method of crushing various substances into fine particles using any of such apparatuses. More particularly, the present invention provides such apparatuses and methods wherein substances can be mixed and/or crushed into fine particles without having to use the mechanical power.

Any of the apparatuses and methods according to the present invention may be utilized in applications, such as those involving the process of crushing the foodstuffs and raw medicinal substances into fine particles, the process of deactivating/sterilizing any enzymes/spores contained in the foodstuffs and raw medicinal substances, the process of deodorizing the substances, the process of rendering industrial wastes harmless, and any other like processes.

2. Prior Art

A typical conventional stationary-type mixing apparatus that can be operated without having to use the mechanical power is disclosed in the Japanese patent application as published under No. S58 (1983)-13822. This stationary mixing apparatus is shown in FIG. 59(a) through FIG. 59(c), and comprises a cylindrical casing 203 having an inlet 201 on one end and an outlet 202 on the opposite end, and a plurality of fluid conducting units provided inside the cylindrical casing 203. Each of the fluid conducting units includes a larger-diameter round plate 206 and a smaller-diameter round plate 207 that are arranged coaxially such that they are placed adjacently to each other. In each fluid conducting unit, the larger-diameter round plate 206 and smaller-diameter round plate 207 have a plurality of polygonal (multi-sided) cells 204, 205 on the respective sides facing opposite each other. The cells 204, 205 on each of the round plates 206, 207 are arranged like a honeycomb, and each of the cells 204, 205 has its front side open.

The larger-diameter round plate 205 has a diameter that matches the inner diameter of the cylindrical casing 203, and has a fluid flow hole 208 through the center thereof.

The larger-diameter round plate 206 and smaller-diameter round plate 207 are arranged such that the open front sides of the cells 204 on the larger-diameter round plate 206 are located to face opposite the open front sides of the corresponding cells 205 on the smaller-diameter round plate 207, the cells 204 on the larger-diameter round plate 204 being placed in alternate positions with regard to the corresponding cells 205 on the smaller-diameter round plate 205 and with each of the cells on the one round plate, that is, the larger or smaller round plate, being capable of communicating with at least one or more of the cells on the other round plate, that is, the smaller or larger round plate. Thus, each individual fluid conducting unit may include one such larger-diameter round plate 206 and one such smaller-diameter round plate 207 that are disposed to face opposite each other.

It is shown in FIG. 59(a) that a number of such individual fluid conducting units are installed within the cylindrical casing 203, wherein in any two adjacent fluid conducting units, the round plates of the same diameter, that is, the larger-diameter round plates or smaller-diameter round plates, are placed to face opposite each other.

There is installed one fluid conducting unit on each of the opposite ends of the cylindrical casing 206, and each of the fluid conducting units on the opposite ends has its larger-diameter round plate located on the respective end side, so that it can communicate with the inlet 201 or outlet 202 through the respective center fluid flow hole 208.

Substances being processed, such as being mixed, usually has the form of a fluid, which may be introduced under the applied pressure through the inlet 201 into the space inside the cylindrical casing 203. The fluid may then flow through the fluid flow hole 208 in the first fluid conducting unit located on the upstream side, flowing into the cylindrical casing 203. The fluid that has passed through the larger-diameter round plate 206 in the first fluid conducting unit may flow toward the smaller-diameter round plate 207 in the first fluid conducting unit, where the fluid is prevented from flowing in the straight direction. When passing through the cells 205, 204 on the smaller-diameter round plates 205, 204 in the first and second fluid conducting units that are located adjacently to each other, the fluid must thus change its direction, going radially from the center toward the outer circumferential side. Then, the fluid goes toward the smaller-diameter round plate 207 in the third fluid conducting unit located downstream of the second fluid conducting unit, where the fluid passes through the gap between the smaller-diameter round plate 207 and the inner circumferential wall of the cylindrical casing 203, flowing toward the cells 205 on the smaller-diameter round plate 207 in the fourth fluid conducting unit located downstream of the third fluid conducting unit and then flowing toward the larger-diameter round plate 206 in the fourth fluid conducting unit where the fluid is prevented from flowing in the straight direction, changing its direction. Then, the fluid passes through the cells 205, 204 in the third and fourth fluid conducting units that communicate with each other, flowing from the outer circumferential side toward the center. Then, the fluid passes through the fluid flow hole 208 in the larger-diameter round plate on the fourth fluid conducting unit, flowing toward the cells 204 on the larger-diameter round plate 206 in the fifth fluid conducting unit located downstream of the fourth fluid conducting unit. This is repeated until the fluid reaches the final fluid conducting unit, where the fluid goes out of the cylindrical casing 203 through the outlet 202.

It may be understood from the above description that when the fluid passes through the cells 204 on the larger-diameter round plates in the adjacent fluid conducting units that face opposite each other and then through the cells 205 on the smaller-diameter round plates on the adjacent fluid conducting units that face opposite each other, those cells may have the effect of dispersing, reversing or swirling the flow of the fluid. That is, the fluid may be diffused or dispersed, flowing radially from the center toward the outer circumferential side and from the outer circumferential side toward the center. This flow may be repeated alternately until the fluid can be mixed completely. In this way, the mixing process can occur efficiently.

In the prior art mixing apparatus described above, wherein a plurality of polygonal (multi-sided) cells 204, 205 are provided like a honeycomb on each of the larger and smaller round plates, but those cells 204a, 205a that are located on the respective outer peripheral sides have the polygonal shapes that are not similar to those of the cells 204, 205, that is, one or two sides are lacking from the polygonal shape. When the fluid flows through those cells having one or two sides lacking, the flow may tend to be focused upon those cells. This means that the fluid would only flow through those cells. Instead, all possible routes through which the fluid can flow will be lost. This may be referred to as the “short path” effect. Thus, the fluid might collide with those cell walls, causing the fluid to have the complex dispersing, reversing, swirling, radially dispersing and focusing actions, which may be repeated. This would reduce the mixing efficiency. The problems that have been described so far are encountered by the stationary mixing apparatus disclosed in the Japanese patent application No. 58 (1983)-133822.

For the round plate having the circular shape such as the one that is disclosed in the Japanese patent application No. 58 (1983)-133822, it is very difficult to arrange the cells on any two adjacent round plates opposite each other such that the cells on one round plate can be placed in alternate positions with regard to the cells on the other round plate, with each of the cells on one round plate being able to communicate with at least one or more of the cells on the other round plate. In this case, it may be possible to provide guide pins for arranging the cells in the appropriate positions, but some of the cells must be eliminated because of the presence of the guide pins. This means that the number of the cells must be reduced As described above, this might also cause the fluid to collide with the cell walls, causing its flow to have the complex dispersing, reversing, swirling, radially dispersing, and focusing actions. Repeating those actions would affect the mixing efficiency.

It should be noted that the round plate 206 such as the one that is employed in the Japanese patent application No. 58(1983)-138822 has its outer diameter that matches the inner diameter of the casing 203 so that the round plate can provide the sealing function. This makes it difficult to move the round plate 206 into or out of the casing 203. To avoid this, the inner diameter of the casing 203 may be larger by the thickness of the seal, thereby blocking the flow of the fluid. The casing 203 must be long enough to accommodate a plurality of fluid conducting units, and each of the fluid conducting units must be sealed along the entire length of the casing 203. If the pressure under which the fluid is supplied is increased, it may break some of the seal, creating a partial gap between the outer diameter of the round plate 206 and the inner diameter of the casing 203. If such situation occurs, the fluid may take the short path through the gap along the entire length of the casing 203, flowing toward the outlet 202 without having the required mixing action, and the fluid may go out of the casing 203 through the outlet 202. This would affect the uniform mixing efficiency.

It may be appreciated that the stationary mixing apparatus as disclosed in the Japanese patent application No. 58(1983)-133822 is only designed to mix substances. Thus, the apparatus cannot provide the capabilities of crushing the substances into fine particles or improving the quality of the substances as well as mixing the substances.

For example, there is a conventional method of crushing a fluid of particular substances into ultrafine particles having a particle size of between about 1 nm and 0.1 μm, wherein any ingredient extracting element and raw substances may be evaporated by thermal plasma or like into a particular gas within a reactor vessel, from which ultrafine particles may be generated by causing the raw substances to react with the gas. According to this method, the ultrafine particles may be obtained by the long-time, continuous process in which the gas that contains the ultrafine particles in the floating and dispersing state within the reactor vessel may be introduced into a recovery vessel through a heat exchanger equipped with a cooling pipe where the ultrafine particles may be cooled by the coolant, and the ultrafine particles within the recovery vessel may be passed through a filter and collected.

The apparatus that may be used in conjunction with the above method includes the reactor vessel and recovery vessel for implementing the method, and therefore must have a larger construction. The apparatus can be running continuously for a relative long time, but the ultrafine particles that have been collected into the recovery vessel by passing them through the filter will have produced the secondary and tertiary coagulations as some time elapses. Thus, the ultrafine particles will gather into gross particles, or will have the temperature that exceed the heat resistant temperature tolerance of the machine components that are located upstream of the recovery vessel. When this occurs, the illumination of the thermal plasma must be suspended for a certain time so that the fine particles can be recovered and the machine components can be disassembled and cleaned. Then, the machine components must be reassembled. This process is laborious and inefficient.

There is also another conventional apparatus and method. According to this apparatus and method, a circulating fluid that contains any ingredient extracting element and raw substances being processed may be jetted into a storage tank through a jet nozzle, allowing the resulting atomized particles in the storage tank to flow by the inertia action through the fluid flow path that is placed under a low pressure, and allowing the fine particles in the atomized particles to flow through the fluid flow path, and then to be attracted through the outlet of the fluid flow path into the particle capturing means that is placed under the low pressure reduced by the decompressor pump. The fine particles that have been captured into the particle capturing means under the low pressure may expand themselves, and therefore must be classed into different sizes by allowing the fine particles to pass through the primary and secondary filters in the gross particle capturing means, and must then be captured by the gross particle capturing means.

The apparatus that may be used in conjunction with the method described above must have a large construction accordingly. The jet nozzle may become clogged with the mixture of finely crushed particle substances and liquid (circulating fluid). Each time the jet nozzle becomes clogged, the machine components must be disassembled, cleaned and reassembled. As the apparatus is only designed to provide the particular fine particles that are desired to be obtained, the supply of the raw substances being crushed into fine particles must be increased. Thus, the wastes that may result from the ingredient extracting process will also be increased. The disposal of those wastes will raise the environmental problem.

There are also several conventional methods for promoting or retarding the dissolving process of some environment polluting substances such as substances that are difficult to dissolve and the progress of the chemical reaction of such substances with any gaseous reacting substances or solid reacting substances, or for promoting or retarding the generating and dissolving process of some chemical substances by controlling the chemical reaction. Some methods take advantage of the ultracritical processing, and other methods take advantage of the electromagnetic waves, ultrasonic waves, infrared rays, far infrared rays and the like.

For the method that takes advantage of the ultracritical processing, the substances that are subject to the ultracritical processing may be crushed into fine particles, which may then be mixed into a fluid. Then, the fluid may be introduced into the reactor vessel that is placed under the ultracritical conditions by increasing the internal temperature and pressure up to a particular numerical level. Then, any gaseous oxidizer or liquid oxidizer such as air, oxygen, carbon dioxide and the like is forced into the reactor vessel where the oxidizing dissolution reaction or other actions can occur.

Dissolving the substances being dissolved and their chemical reaction with any reaction substances may essentially consist of dissolving the substances by causing their molecules to collide with each other and then mixing them together. Thus, the substances must firstly be introduced into the respective reactor vessels, where they must then be mixed together.

It should be appreciated, however, that when the apparatus is placed under the ultracritical conditions in which the internal pressure is set to a high level, problems may occur with the power required to drive the apparatus for mixing or stirring the substances fed into the apparatus or on the seals provided in the reactor vessel. The apparatus that may be used in conjunction with the above method must have a large construction, and the desired processing cannot be performed satisfactorily.

In the method that takes advantage of the electromagnetic waves for dissolving the substances that are hard to dissolve, the raw substances may be introduced into the reactor vessel that is heated by induction, where the reaction and dissolving process for the substances may be promoted by heating the substances by the induction heating as well as by the electromagnetic wave heating by the electromagnetic waves that are produced by the induction heating.

For the method that takes advantage of the ultrasonic waves for dissolving the substances hard to be dissolved, any liquid substance and any gaseous substance being processed may be fed into the reactor vessel, where those substances may be exposed to the ultrasonic waves coming from the ultrasonic wave source in the reactor vessel so that the optimal mixing conditions or quick reaction speed can be obtained.

For the method that takes advantage of the infrared rays or far infrared rays for generating and/or dissolving any chemical substances, the chemical reaction may be controlled by illuminating the infrared rays or far infrared rays into the reactor vessel so that the generation and dissolving process for the substances can be promoted or retarded.

### SUMMARY OF THE INVENTION

In light of the problems associated with the prior art that has been described above, the present invention proposes to provide apparatuses and methods that will be described below in specific details.

One object of the present invention is to provide a mixing apparatus that provides the uniform mixing capability that has been enhanced by adding an appropriate improvement to the prior art stationary mixing apparatus described above.

Another object of the present invention is to provide a mixing apparatus that adds an improvement to the prior art stationary mixing apparatus described above, so that it can contain machine components that are easy to be assembled, including the cylindrical casing having the inner surface that can be worked with ease, and can thus reduce the total manufacturing or assembling costs.

A further object of the present invention is to provide a mixing apparatus that adds an improvement to the prior art stationary mixing apparatus described above, so that it can prevent any possible fluid leaks that would occur if any partial gap is present, and can also prevent the non-uniform mixing that would occur if the fluid takes the shortcut path (short path).

Still another object of the present invention is to provide a mixing apparatus that adds an improvement to the prior art stationary mixing apparatus described above, such that it can provides the mixing and/or crushing capabilities for crushing substances such as foodstuffs and medicinal substances, or even the substances that are hard to dissolve such as environment polluting substances, into finely crushed spherical particles having the particle sizes of between about 1 nm and 1 μm.

There is no mixing apparatus that has a comparatively small size, and despite the small size, is capable of producing and/or creating fine particles by allowing a fluid to flow under the applied pressure through the fluid flow path formed by the cells each having its front side open.

In view of the above fact, it is a further object of the present invention to provide a mixing apparatus that adds an improvement to the prior art stationary mixing apparatus described above, such that despite its small size, it can provide the mixing and/crushing capabilities for crushing substances into fine particles, more specifically, crushing various types of fibrous substance into fine particles.

Any of the apparatuses provided by the present invention may be used as a heat exchanger, and may also be used to dissolve such substances or promote the chemical reaction for those substances by taking advantage of the critical process, ultracritical process, electromagnetic waves, ultrasonic waves, infrared rays, far infrared rays and the like.

The present invention also proposes to provide several methods that may be implemented by any of the mixing apparatuses mentioned above for crushing substances into fine particles. Any of the methods proposed by the present invention may be used in conjunction with the critical process or ultracritical process or other processes, such as the substance dissolving, chemical reaction promoting and the like, that take advantage of the electromagnetic waves, ultrasonic waves, infrared rays, far infrared rays and the like.

The processes, such as the substance dissolving, chemical reaction promoting and like processes, which may be implemented by the methods of the present invention by taking advantage of the electromagnetic waves, ultrasonic waves, infrared rays, far infrared rays and the like may provide the desired processing results, such as the improved substance surface activity, improved substance quality, promoted chemical promotion and improved substance dissolving.

Any of the apparatuses and methods provided by the present invention permits the crushing, reaction promotion, dissolving and mixing processes for the substances to occur continuously so that the consistent processing results can be obtained, without having to use the large scale machine.

In order to accomplish the above objects, the apparatus according to the present invention is designed to mix and/or crush substances into fine particles, and comprises a cylindrical casing having a hollow portion therein and having an inlet on one end and an outlet on the opposite end, wherein a fluid flow path is provided to run between the inlet and outlet through the cylindrical casing. The fluid flow path has the unique structural features that will be described below.

Firstly, the fluid flow path may be formed by a first structural member having a plurality of first cells thereon, each having its front side open, and a second structural member having a plurality of second cells thereon, each having its front side open, wherein the first structural member and second structural member are arranged adjacently to each other such that the first cells on the first structural member and second cells on the second structural member have the respective open front sides placed to face opposite each other and in alternate positions with regard to each other, with each of the cells on one structural member communicating with at least one or more of the cells on the other structural member.

The open front side of each of the first cells and second cells has the shape that may be defined by the shape surrounded by line segments P-S-Q-S2-R-S1-P, where when an imaginary right-angled isosceles triangle ABC, where A refers to a vortex and BC refer to two equal sides, is given, S refers to any arbitrary point other than points A, R that is located along the line segment A-R connecting between vortex A and midpoint R on the base B-C, P refers to any arbitrary point other than points A, B that is located along the hypotenuse A-B, and Q refers to any arbitrary point other than points A, C that is located along the hypotenuse A-C and where S1 refers to the point where the line segment P-S will intersect with the base B-C when the line segment P-S connecting between points P and S is turned about the point P, and S2 refers to the point where the line segment Q-S will intersect with the base B-C when the line segments Q-S connecting between points Q and S is turned about the point Q.

When the fluid flow path is formed by the first structural member having a plurality of first cells thereon, each having its front side open, and the second structural member having a plurality of second cells thereon, each having its front side open, wherein the first structural member and second structural member are arranged adjacently to each other such that the first cells on the first structural member and second cells on the second structural member have the respective open front sides placed to face opposite each other and in alternate positions with regard to each other, with each of the cells on one structural member communicating with at least one or more of the cells on the other structural member, the space on the front sides of the cells facing opposite each other may be divided by at least one or more cells on the other structural member. Then, the area and volume where the space is divided on the open front sides of the cells are different before and after the fluid flow path, respectively. Specifically, the fluid flow path that is formed by a plurality of structural members such that any two adjacent structural members are arranged opposite each other, with the open front sides of the cells on one structural member facing opposite the open front sides of the cells on the other structural member, with the cells on one and the other structural members being arranged in alternate positions with regard to each other, and with each of the cells on one structural member communicating with at least one or more of the cells on the other structural member. The fluid flow path runs from the inlet toward outlet of the cylindrical casing, and includes a plurality of continuous divisional sections whose areas and volumes are different before and after the location where each divisional section exists along the continuous fluid flow path. When a fluid of substances being processed, such as being mixed and/or being crushed, is fed into the fluid flow path, the fluid may cause the colliding, reversing, swirling, radial dispersing, and gathering motions each time the fluid passes continuously through the divisional sections each having the different area and volume. Those motions may be repeated until the fluid reaches the end of the fluid flow path. More specifically, when the fluid passes through one divisional section having the area and volume different from those of another divisional section, on one hand, the fluid may have the coagulation action that is increased by the embracing pressure. When the fluid passes through one divisional section having the area and volume different from those of another divisional section, on the other hand, the fluid may be released from the embracing pressure at once, and may then have the dissolving and crushing actions. The fluid can be mixed uniformly and/or crushed into very fine particles having the desired size, such as nearly pure spherical forms, by repeating the above motions and actions.

The apparatus according to the present invention includes the first structural member having a plurality of first cells thereon, each having its open front side formed like the particular shape described above, and the second structural member having a plurality of second cells thereon, each having its open front side formed like the particular shape described above, wherein the fluid flow path is formed by the first and second structural members that are arranged adjacently to each other, with the cells on the first structural member facing opposite the cells on the second structural member, with the first cells being placed in alternate positions with regard to the second cells facing opposite the first cells, and each of the cells on one structural member communicating with at least one or more of the cells on the other structural member, and wherein the fluid flow path runs through the cylindrical casing from the inlet toward outlet of the cylindrical casing. Substances that have been crushed into gross particles, as well as liquid, may be mixed into a fluid, and the fluid may be introduced under the applied pressure into the apparatus described above in accordance with the present invention. When the fluid is traveling through the apparatus, pressures may be produced inside and outside the fluid, such as primary embracing pressure, secondary embracing pressure, exploded dispersion, twisting, swelling, kneading and friction. Thus, the gross particle substances may be crushed into ultrafine particles and molecules. In addition, the surface activity improvement, quality improvement, reaction promotion and dissolving can be provided for the ultrafine particles and molecules in the consistent and continuous manner.

In the above description, it should be noted that the line segments P-S and Q-S may be formed as a single straight line, or a broken line consisting of several straight lines. It may also be formed as a sine curve, arc curve and the like. Otherwise, it may be formed as a combination of a straight line, broken line and curved line.

It should also be noted that the shape of the open front side for the first and second cells, each having its front side open, may be determined by setting point P and point Q as the midpoint along the hypotenuse A-B and the midpoint along the hypotenuse A-C, respectively.

The area of the shape surrounded by the line segment P-S-Q-S2-S1-P thus obtained may correspond to one half the area of the imaginary original right-angled isosceles triangle.

This may be explained more clearly by referring to FIG. 1. In FIG. 1, point P and point Q may be located on the midpoint along the hypotenuse A-B and on the midpoint along the hypotenuse A-C, respectively. Then, the points where the respective perpendiculars dropped from point P and point Q intersect with the base B-C are designated as S3 and S4, respectively. In this case, the area of the rectangle P-S3-R-S4-Q-P may correspond to one half the area of the right-angled isosceles triangle ABC.

It may be appreciated from the above description that the point S1 is the point where the line segment P-S will intersect with the base B-C when the line segment P-S is turned about the point P, and the point S2 is the point where the line segment Q-S will intersect with the base B-C when the line segment Q-S is turned about the point Q. It may be seen from FIG. 1 that the area of the portion denoted by S5 in FIG. 1 will be equal to the area of the portion denoted by S6, and the area of the portion denoted by S7 will be equal to the area of the portion denoted by S8. As a result, the area of the shape surrounded by the line segment P-S-Q-S2-S1 drawn as described above will be equal to one half the area of the imaginary original right-angled isosceles triangle ABC.

In the apparatus described above, the shape of the open front side for the first cells and second cells, each having its front end open, may be determined by setting points P and Q on the midpoint along the hypotenuse A-B and on the midpoint along the hypotenuse A-C. Thus, the fluid flow path may be formed by the continuous sequence of divisional sections, each of which has the area and volume that are different from those of any other divisional spaces before and after the divisional section. When a fluid of substances being mixed and/or crushed is fed under the applied pressure into the fluid flow path, passing through the fluid flow path, the fluid may have the uniform mixing process and/or may have the crushing process in which the substances may be crushed into fine particles that have the desired spherical forms, such as the nearly pure spherical forms. In addition to this effect, other advantageous effects may be obtained as described below.

As described above, the shape of the open front side for the first cells and second cells may be defined by placing points P and Q on the midpoint along the hypotenuse A-B and on the midpoint along the hypotenuse A-C, respectively, the first structural member having the plurality of first cells thereon, each having its front side open, and the second structural member having the plurality of second cells thereon, each having its front side open, can have the first cells and second cells arranged on the respective entire surfaces thereof, without any gaps being produced between any adjacent cells, respectively.

FIGS. 1 through 13, FIG. 31(a), FIG. 32(a), FIG. 33(a), and FIG. 53 represent examples of the shapes for the open front sides of the first and second cells that are arranged on the first and second structural members, respectively, in which each of the cells has the right-angled isosceles triangle as the basic shape as defined above.

It may be seen from those figures that each of the first cells and second cells having its front side open has the portion that is surrounded by the shape surrounded by the line segment P-S-Q-S2-S1-P1 and the shape that is similar to, but is smaller or larger than, the above shape, and that portion may be raised as the wall on the first structural member and second structural member, respectively. FIG. 31(b), FIG. 32(b) and FIG. 33(b) represent examples of the first cells and second cells, each having its front side open, in which each cell has the portion that is surrounded by the shape surrounded by the line segment P-S-Q-S2-S1-P1 and the shape that is similar to, but is smaller or larger than, the above shape, and that portion may be raised as the wall on the first structural member and second structural member, respectively.

It may be understood from the foregoing description that the area of the shape surrounded by the line segment P-S-Q-S2-S1-P should be equal to one half the area of the imaginary original right-angled isosceles triangle ABC. In this way, the first structural member having the plurality of first cells thereon, each having its front side open, and the second structural member having the plurality of second cells thereon, each having its front side open, can have the first cells and second cells arranged over the entire surfaces thereof, respectively, without any gaps being produced between any adjacent cells. If the above conditions are met, the line segment S2-S1 in the line segment P-S-Q-S2-S1-P has not to be the straight line on the base B-C.

In the apparatus of the present invention described above, for example, the shape of the open front side for the first and second cells, each having its front side open, may also be defined by placing point P and point Q on the point symmetry location with the line segment A-R as the center on the hypotenuses A-B, A-C, respectively, and by making either the line segment S2-R or R-S1 in the line segment S-2-R-S1 into another line segment having any shape different from the straight line on the base B-C and then making the line segment as the point symmetry to the line segment having the above any shape into the other segment, with the midpoint R as the center.

FIG. 14 represents an example of the above case, in which point P and point Q are the midpoints on the hypotenuses A-B and A-C, respectively. In this example, the area of the portion denoted by S9 will be equal to the area of the portion denoted by S10. Thus, the shape of the open front side for the first cells and second cells, each having its front side open, can be defined as efficiently as the case where point P and point Q are the midpoints on the hypotenuses A-B and A-C, respectively.

In the apparatus of the present invention described above, it should be noted that the fluid flow path may be formed in the axial direction of the cylindrical casing or in the direction perpendicular to the axial direction of the cylindrical casing. For example, the fluid flow path may be provided in the axial direction of the cylindrical casing, if the sides on which the open front sides for the plurality of first cells on the first structural member and the open front sides for the plurality of second cells on the second structural member face opposite each other extend in the axial direction of the cylindrical casing, while the fluid flow path may be provided in the direction perpendicular to the axial direction of the cylindrical casing, if the sides opposite each other extend in the direction perpendicular to the axial direction of the cylindrical casing.

For example, the first structural member and second structural member may be mountable within the cylindrical casing, and the fluid flow path may be provided in the axial direction of the cylindrical casing or in the direction perpendicular to the axial direction of the cylindrical casing. It should be understood that there are several ways in which the first structural member and second structural member may be mountable within the cylindrical casing. For example, one way is to provide recesses around the inner circumferential wall of the cylindrical casing, and construct the first and second structural member so that they can be fitted in those recesses. When the cylindrical casing and the first and second structural members are constructed in this way, it would become easier to work the inner circumferential wall of the cylindrical casing, and assemble the cylindrical casing, first structural member and second structural member into the complete apparatus. This construction provides the advantage in that it ensures that no partial gaps would occur between the cylindrical casing and the first structural member and second structural member. That is, the short path would be prevented.

As a variation of the apparatus of the present invention described above, the cylindrical casing may be formed by the first structural member so that the plurality of first cells, each having its front side open, can be provided on the inner circumferential wall of the first structural member forming the cylindrical casing, and the plurality of second cells, each having its front side open, can be provided on the outer circumferential wall of the second structural member that may be mounted inside the first structural member forming the cylindrical casing. In this way, the fluid flow path may be provided in the axial direction of the cylindrical casing formed by the first structural member. As an alternative form of the fluid flow path, which is formed by placing the first and second structural members such that the cells on the first structural member can face opposite the cells on the second structural member face, the fluid flow path may be formed by permitting the second structural member having a particular outer circumferential diameter to be fitted into the cylindrical casing formed by the first structural member having the inner circumferential diameter that matches the outer circumferential diameter of the second structural member. For example, the fluid flow path may be formed by permitting the second structural member simply to be fitted in the recess provided on the cylindrical casing formed by the first structural member. In this variation, the first structural member forming the cylindrical casing and the second structural members can be worked easily so that they can provide the inner circumferential surface and outer circumferential surface, respectively, and that those members can be assembled into the complete machine. This variation may also provide the advantage in that it ensures that no partial gaps would occur between the cylindrical casing and the first structural member and second structural member. That is, the short path would be prevented.

As a further variation of the above variation, the first structural member forming the cylindrical casing may have the divisible construction, and the mounting and removing of the second structural member within and from the cylindrical casing may be performed by dividing the cylindrical casing. This makes it easy to mount the second structural member within the cylindrical casing formed by the first structural member, and assemble those members into the complete machine. This may also provide the easy maintenance of those members. It may be appreciated that the cylindrical casing may have the divisible construction or form that permits the cylindrical casing to be divided in the axial direction thereof.

As a variation of the apparatus described above, the first structural member and the second structural member may comprise of a first plate member having a plurality of cells, each having its front side open, on one side thereof, or a second plate member having a plurality of cells, each having its front side open, on both of the opposite sides thereof, wherein the first and second plate members are mounted inside the cylindrical casing so that the fluid flow path can be formed in the axial direction of the cylindrical casing or in the direction perpendicular to the axial direction of the cylindrical casing. For example, recesses may be provided on the inner circumferential wall of the cylindrical casing, and the first and second plate members may have the form or construction that permits the first and second plate members to be fitted in the recesses on the cylindrical casing. Then, the fluid flow path may be formed between the first and second plate members that are placed to overlap each other tightly. This makes it easy to work the cylindrical casing formed by the first plate member so that it can provide the inner circumferential surface, and to assemble those members into the complete machine. This may also provide the advantage in that it ensures that no partial gaps would occur between the cylindrical casing and the first plate member and second plate member. That is, the short path would be prevented.

It should be noted that the plurality of cells, each having its front side open, on the other side of the second plate member may be provided by rotating the cells on the one side of the second plate member through a predetermined angle, such as 45 degrees, 90 degrees, or 180 degrees on the locations of the plurality of cells, each having its front side open, on the other side of the second plate member corresponding to the locations of the plurality of cells, each having its front side open, on the one side of the second plate member.

As one alternative form, the plurality of cells, each having its front side open, on the other side of the second plate member may be provided in the locations of the cells on the other side of the second plate member that are different from the locations of the plurality of cells, each having its front side open, on the one side of the second plate member.

As a further variation, the plurality of cells, each having its front side open, on the other side of the second plate member may be provided by rotating the cells on the one side of the second plate member through a predetermined angle, such as 45 degrees, 90 degrees, or 180 degrees on the locations of the plurality of cells, each having its front side open, on the other side of the second plate member that are different from the locations of the plurality of cells, each having its front side open, on the one side of the second plate member.

In any of the forms described above, when the first and structural members are placed to adjoin each other tightly such that the cells, each having its front side open, on the first structural member can face opposite the cells, each having its front side open, on the second structural member, or when the two structural members are placed to adjoin each other tightly such that the cells on the one and other structural members can face opposite each other, the cells on the one structural member can be placed in alternate positions with regard to the cells on the other structural member, and each cell on the one structural member can communicate with at leas one or more of the cells on the other structural member.

As described above and as shown in FIGS. 1 through 14, each of the cells has the right-angled isosceles triangle as its basic shape, and those cells are arranged such that their respective open front sides face opposite each other, with the cells facing opposite each other being placed in alternate positions, and each of the cells on one structural member can communicate with at least one or more of the cells on the other structural member. When the fluid flow path is thus formed, the space that may be created on the open front sides of those cells can have many different configurations and forms (area, volume). When the fluid flows through the fluid flow path, it can manifest the more complicate motions that are caused by the colliding, dispersing, reversing and swirling actions of the fluid. Thus, the mixing and/or crushing process can be promoted.

As a variation of the apparatus described above, a plurality of frame members may be provided on the upstream and downstream sides of the location where the fluid flow path is formed by the first and second structural members inside the cylindrical casing, wherein those frame members have openings arranged like a honeycomb and communicating with each other in the axial direction of the cylindrical casing, and are placed to overlap each other so that the openings on the adjacent frame members facing opposite each other can be placed in alternate positions with regard to each other in the direction perpendicular to the axial direction of the cylindrical casing.

In the above variation, when the fluid flows through the fluid flow path in the cylindrical casing, it can have the complicate motions that are caused by the flow path formed by the openings of the frame members before and after the fluid flow path, as well as the complicate motions that are caused by the colliding, dispersing, reversing and swirling actions of the fluid. The mixing and/or crushing process can be promoted further.

In this variation, the cylindrical casing may also have the divisible construction. For example, it may be divisible in the axial direction thereof, and the first structural member including the first plate member and the second structural member including the second plate member may be mounted inside the cylindrical casing and removed from the cylindrical casing, by dividing the cylindrical casing.

In the embodiment in which the frame members are employed, the cylindrical casing may have the divisible construction, for example, it may be divided into two parts in the axial direction thereof, and the first structural member including the first plate member and the second structural member including the second plate member, as well as the first and second frame members arranged to overlap each other, may be mounted inside the cylindrical casing and removed from the cylindrical casing by dividing the cylindrical casing into the two parts.

This embodiment permits the first and second plate members as well as the frame members to be mounted inside the cylindrical casing with ease, making it easy to assemble, disassemble, and reassemble those members for the maintenance purposes.

In the apparatus described above in accordance with the various embodiments of the present invention, the plurality of first cells, each having its front side open, on the first structural member and the plurality of second cells, each having its front side open, on the second structural member should desirably have the identical shape. This provides the advantage in that when the first structural member and second structural member are arranged such that the cells, each having its front side open, on the first structural member and the cells, each having its front side open, on the second structural member can face opposite each other, by using the simple way of rotating the first cells through an angle of 45 degrees, 90 degrees or other degrees, on the locations of the second cells on the second structural member that correspond to the locations of the first cells on the first structural member and then providing the second cells on the second structural member, the cells facing opposite each other can be placed in alternate positions with regard to each other, with each of the cells on one structural member communicating with at least one or more of the cells on the other structural member. This may also provide the advantage in that the first cells and the corresponding second cells can have the regular size, which prevents the short path from occurring.

In the apparatus described above in accordance with the various embodiments of the present invention, the plurality of first cells, each having its front side open, on the first structural member and/or the plurality of second cells, each having its front side open, on the second structural member may be arranged on any location on the first and second structural members, respectively, as long as the first and second structural members are arranged such that they can form the fluid flow path as described above. It should be noted, however, that the fluid flow path may also be formed by arranging the first cells and second cells like the honeycomb on the first and second structural members, respectively.

In this honeycomb arrangement, the shape of the open front side for the first and second cells can be defined by placing point P and point Q on the midpoint along the hypotenuses A-B and A-C of the imaginary right-angled isosceles triangle, respectively. In this way, the first and second cells can be arranged on the entire surface of any side of the first and second structural members, respectively, without producing any gaps between the adjacent cells.

In the apparatus described above in accordance with the various embodiments of the present invention, an inlet space and an outlet space may be provided on the upstream side and downstream side of the location where the fluid flow path is formed by the first and second structural members, wherein the inlet space has the conical shape whose diameter is increasing from the inlet toward the downstream, and the outlet space has the conical shape whose diameter is decreasing toward the outlet. This variation may provide the advantage in that when the fluid is flowing through the cylindrical casing, it can have the motions, such as dispersing, concentrating and colliding, before and after the fluid flow path, as well as the complicate motions, such as colliding, dispersing, reversing and swirling, which are caused as the fluid flows through the fluid flow path. Thus, the mixing and/or crushing process can be promoted.

In this case, it should be noted that when the fluid flow path between the inlet space and outlet space, each having the conical shape, is being formed between the inner circumferential wall of the inlet space and the outer circumferential wall of the outlet space, the corresponding structural members having the conical shape may be disposed in the inlet space and outlet space.

As a variation of the apparatus described above in accordance with the various embodiments of the present invention, the first structural member may be the cylindrical casing, and the plurality of first cells, each having its front side open, which are provided on the first structural member in accordance with the previous embodiments may be provided on the inner circumferential wall of the cylindrical casing and the plurality of second cells, each having its front side open, which are provided on the second structural member in accordance with the previous embodiments may be provided on the outer circumferential wall of the structural member that is mounted inside the cylindrical casing. In this variation, the fluid flow path may be formed between the inner circumferential wall of the cylindrical casing and the outer circumferential wall of the structural member mounted inside the cylindrical casing. In the manner described above, when the fluid flow path between the inlet space and outlet space, each having the conical shape, is being formed between the inner circumferential wall of the inlet space and the outer circumferential wall of the outlet space, the corresponding structural members having the conical shape may be disposed in the inlet space and outlet space. This provides the advantage in that the fluid can flow smoothly through the fluid flow path formed between the inner circumferential wall and the outer circumferential wall of the structural member mounted inside the cylindrical casing.

In any of the embodiments of the apparatus according to the present invention that have been described so far, the first structural member and/or second structural member may be made of any one selected from the group consisting of carbons or composite metal materials such as combinations of carbon and other metals, ceramics, and other minerals.

The cylindrical casing may also be made of any one selected from the group consisting of carbons or composite metal materials such as combinations of carbon and other metals, ceramics, and other minerals.

The first structural member and/or second structural member may also be made of any one of natural resins and synthetic resins.

Similarly, the cylindrical casing may also be made of any one of natural resins and synthetic resins.

The first structural member, second structural member and cylindrical casing may also be made of any of the thermal conductive materials such as copper, aluminum, carbon and the like, in which case the apparatus including such components may also be used as the heat exchanger.

It should be appreciated that the first structural member, second structural member and cylindrical casing may also be made of any of SUS metals.

In any of the embodiments of the apparatus according to the present invention that have been described so far, magnets may be disposed on the outer circumferential wall of the cylindrical casing.

The cylindrical casing may have any cross sectional shapes, such as round, elliptic, and polygonal (triangle, square, etc.). The cylindrical casing may include a central portion that has any cross sectional shapes, such as round, elliptic, and polygonal (triangle, square, etc.), and portions having the conical and pyramid shapes that are provided before and after the inlet and outlet sides, respectively.

In any of the embodiments of the apparatus according to the present invention that have been described so far, the apparatus may be used in conjunction with any one of the ultrasonic wave illuminating device, electromagnetic wave illuminating device, high-frequency wave illuminating device, and laser beam illuminating device that may be linked to the upstream and/or downstream side of the apparatus.

In any of the embodiments of the apparatus according to the present invention that have been described so far, the apparatus may include an inlet that may be linked to the upstream and/or downstream side of the apparatus for feeding some particular types of agents that contain oxygen or alkali agents.

Those agents that are fed through this inlet have the action or effect to cause the oxidization reaction of the substances being processed, or to neutralize any acid generating ingredients that may be contained in the substances being processed.

It may be appreciated that the apparatus according to any of the embodiments described so far permits the different types of substances to be dissolved and/or crushed into ultrafine particle levels or molecule levels although the apparatus has the small-size construction.

When the above mentioned devices are linked to the upstream and/or downstream of the apparatus, the apparatus may be used as the mixing/separating reaction continuous generator.

When the electromagnetic wave illuminating device, infrared ray illuminating device, ultrasonic wave illuminating device and like are linked to the downstream side of the apparatus, or are also linked to the upstream side if required, the process of dissolving the environment polluting substances and the substances hard to be dissolved can be improved remarkably, and the process of generating and dissolving chemical substances can also be improved remarkably.

The method proposed in accordance with one aspect of the present invention for crushing substances into fine particles may be used in conjunction with any of the apparatuses described so far, wherein a fluid in which substances be processed (crushed) are mixed are introduced under the applied pressure through its inlet, and can have the fine particle crushing process while flowing the said fluid from the inlet toward outlet of the cylindrical casing of the apparatus.

The method proposed in accordance with another aspect of the present invention for crushing substances into fine particles may be used in conjunction with any of the apparatuses described so far, wherein a fluid in which substances be processed (crushed) are mixed are introduced introduced under the applied pressure through its inlet, and can have the fine particle crushing process under the continuous critical condition or ultracritical condition while flowing the said fluid continuously from the inlet toward outlet of the cylindrical casing of the apparatus.

As it is used in this specification, the term “critical condition” should be understood to mean that when a particular substance is placed at the higher temperature than the critical temperature of the substance and under the higher pressure than the critical pressure of the substance, the substance is in the gas-liquid coexisting state, not simply in the gas state nor in the liquid state. As it is used in this specification, the term “ultracritical condition” should be understood to means that a particular substance is placed in the state in which it is impossible to tell whether it is in gas state or liquid state when it is above the critical condition. The critical temperature and critical pressure are constant values that are specific to particular types of substances.

Both the critical condition and ultracritical condition for a particular type of substances being processed can be attained, for example, by admixing any gas phase oxidizer or liquid phase oxidizer such as air, oxygen and carbon dioxide with the substances in the form of a fluid, feeding the resulting mixture continuously under the applied pressure into the cylindrical casing so that the fluid can flow from the inlet toward outlet of the cylindrical casing, placing the fluid under the critical condition or ultracritical condition specific to the particular type of substances, and bringing the fluid into the appropriate critical condition or ultracritical condition. It should be noted that the pressure may be controlled by adjusting the applied pressure under which the fluid is fed into the cylindrical casing, and the temperature may be controlled by adjusting the temperature at which the cylindrical casing is heated.

In the method and apparatus according to one aspect of the present invention described above, particular industrial wastes, for example, may be crushed into particles in the form of a fluid. Then, the fluid may be introduced under the applied pressure into the cylindrical casing together with a gas of pure oxygen, and the molecules that are bonded with each other may be dissolved, reduced harmless and discharged from the outlet.

In the method and apparatus according to another aspect of the present invention described above, a mixture of particular substances and a solvent such as carbon dioxide in the form of a fluid may be introduced continuously under the applied pressure into the apparatus, or the cylindrical casing, and may be crushed into fine particles, dissolved, or have the quality improved by placing the fluid continuously under the appropriate critical condition or ultracritical condition.

By using the method and apparatus of the present invention in conjunction with any one of the electromagnetic wave illuminating means, infrared ray illuminating means, far infrared ray illuminating means, and laser beam illuminating means that may be provided on the upstream and/or downstream sides of the cylindrical casing, the process of crushing substances into fine particles, dissolving the substances and improving the quality can be performed more efficiently.

According to the method and apparatus of the present invention, the process of dissolving and improving the quality for the substances hard to dissolve, harmful organic substances, environment polluting substances, chemical substances and the like can be performed efficiently.

In summary, the apparatus for mixing and/or crushing substances into fine particles in accordance with the various embodiments of the present invention includes the fluid flow path, wherein the fluid flow path is formed by arranging a plurality of first cells, each having its front side open, and a plurality of second cells, each having its front side open, such that the open front sides of the first cells and the open front sides of the second cells face opposite each other, with the first cells being placed in alternate positions with regard to the second cells and with each of the first cells communicating with at least one or more of the second cells, and wherein when a right-angled isosceles triangle having two hypotenuses, vortex and base is given, the open front side of each of the first and second cells may be formed by a basic geometric shape having the area that is equal to one half the area of the right-angled isosceles triangle that may be formed by turning line segments connecting any arbitrary points along the two hypotenuses and any arbitrary points along the perpendicular dropped from the vortex to the base clockwise and anticlockwise about the arbitrary points on the hypotenuses as the origins, and intersecting those line segments with the base. The fluid flow path thus formed may includes a continuous sequence of divisional sections, each having the area and volume that are different from those each adjacent divisional section before and after the adjacent divisional section. Then, a fluid of substances being processed may be fed under the applied pressure into the fluid flow path, and may have the internal and external pressures continuously, such as primary embracing pressure, secondary embracing pressure, exploded dispersion, twisting, swelling, kneading, friction and the like, while the fluid is flowing through the continuous sequence of divisional sections having the area and volume different from those of each adjacent divisional section. The process of crushing substances into ultrafine particles and molecules, dissolving substances, and improving the quality of substances can proceed in this way.

By using the apparatus and method of the present invention in conjunction with the electromagnetic wave illuminating device, ultrasonic wave illuminating device, infrared ray illuminating device and the like, the process of dissolving substances hard to dissolve, controlling the chemical reaction for the chemical substances to generate chemical substances, promoting or retarding the dissolving process, improving the surface activity, improving the quality and the like can be performed efficiently.

### BRIEF DESCRIPTION OF THE DRAWINGS

FIG. 1 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 2 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 3 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 4 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 5 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 6 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 7 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 8 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 9 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 10 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 11 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 12 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 13 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention; FIG. 14 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 15 illustrates the apparatus for mixing and/or crushing substances into fine particles in accordance with one embodiment of the present invention, in which FIG. 15(a) represents the cross sectional view with some non-critical parts being not shown, and FIG. 15(b) represents the side elevation of FIG. 15(a);

FIG. 16 illustrates the apparatus shown in FIG. 15(a), in which FIG. 16(a) represents an enlarged plan view showing a first plate member provided in the apparatus, and FIG. 16(b) represents an enlarged plan view showing a second plate member provided in the apparatus;

FIG. 17 illustrates the first and second plate members, in which FIG. 17(a) represents a cross section taken along the line A-A in FIG. 16(a), and FIG. 17(b) represents a cross section taken along the line B-B in FIG. 16(b);

FIG. 18 represents a perspective view of the first plate member shown in FIG. 16(a):

FIG. 19 represents a perspective vies of the second plate member shown in FIG. 16(b);

FIG. 20 represents a perspective view illustrating how the first plate member and the second plate members are placed one over the other;

FIG. 21 illustrates the first and second plate members that are placed one over the other, in which FIG. 21(a) represents a plan view illustrating how the first and second plate members are placed one over the other, and FIG. 21(b) represents a cross section taken along the line C-C in FIG. 21(a);

FIG. 22 represents a perspective view illustrating the outlook of the apparatus for mixing and/or crushing substances into fine particles shown in FIG. 15(a) in accordance with one embodiment of the present invention;

FIG. 23 represents an exploded perspective view illustrating how the components of the apparatus for mixing and/or crushing substances into fine particles are assembled together in accordance with one embodiment of the present invention;

FIG. 24 illustrates the components of the apparatus for mixing and/or crushing substances into fine particles shown in FIG. 23, in which FIG. 24(a) represents a front elevation of the cylindrical casing with a covering mounted thereon, FIG. 24(b) represents a side elevation of FIG. 24(a), FIG. 24(c) represents a cross sectional view illustrating the internal structure of the cylindrical casing with some non-critical parts being not shown, FIG. 24(d) represents a side elevation of FIG. 24(c), FIG. 24(e) represents a front elevation illustrating how the plate members are fitted inside the cylindrical casing, FIG. 24(f) represents a side elevation of FIG. 24(e), FIG. 24(g) represents a cross section of the covering, and FIG. 24(h) represents a side elevation of FIG. 24(g);

FIG. 25 represents a cross sectional diagram illustrating how a fluid flows in the apparatus shown in FIG. 23 for mixing and/or crushing substances into fine particles, with some non-critical parts being not shown;

FIG. 26 is a partly enlarged view of FIG. 25;

FIG. 27 illustrates the apparatus for mixing and/or crushing substances into fine particles in accordance with one embodiment of the present invention, in which FIG. 27(a) represents a cross section with some non-critical parts being not shown, and FIG. 27(b) represents a side elevation of FIG. 27(a);

FIG. 28 illustrates the apparatus for mixing and/or crushing substances into fine particles shown in FIGS. 27(a) and (b), in which FIG. 27(a) represents a front elevation of a structural member that is fitted inside the cylindrical casing of the apparatus with some non-critical parts being not shown, FIG. 28(b) represents a side elevation of FIG. 28(a), and FIG. 28(c) represents a cross section taken along the line D-D in FIG. 28(a);

FIG. 29 illustrates how the components of the apparatus for mixing and/or crushing substances into fine particles shown in FIGS. 27(a) and (b) are assembled together, in which FIG. 29(a) represents a front elevation of the structural member fitted inside the cylindrical casing with some non-critical parts being not shown, and FIG. 29(b) represents a front elevation of the cylindrical casing with some non-critical parts being not shown;

FIG. 30 is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention;

FIG. 31(a) is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention, and FIG. 31(b) is a perspective view of the cell having the shape defined in FIG. 31(a);

FIG. 32(a) is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention, and FIG. 32(b) is a perspective view of the cell having the shape defined in FIG. 32(a);

FIG. 33(a) is a diagram that illustrates how the shape of a cell having one front side open is defined so that the cell can be formed on the surface of a structural member in order to provide a fluid flow path in the apparatus for mixing and/or crushing substances into fine particles in accordance with one embodiment of the present invention, and FIG. 33(b) is a perspective view of the cell having the shape defined in FIG. 33(a);

FIG. 34(a) is a plan view of a plate member having the cells thereon, each of which has its front side open and has the shape defined in FIGS. 31(a) and (b), and FIG. 34(b) is a side elevation of FIG. 34(a);

FIG. 35(a) is a plan view of a plate member having the cells on both sides thereof, each of which has its front side open and has the shape defined in FIGS. 31(a) and (b), and FIG. 35(b) is a side elevation of FIG. 35(a);

FIG. 36(a) is a cross sectional view of the apparatus for mixing and/or crushing substances into fine particles having a fluid flow path formed by the plate members shown in FIGS. 35(a) and (b) in accordance with one embodiment of the present invention, and FIG. 36(b) is a side elevation of FIG. 36(a);

FIG. 37 is a diagram that illustrates how the fluid flow occurs through its flow path in the apparatus shown in FIG. 36;

FIG. 38 presents a microscopic picture of a fluid that has been obtained before the experimental testing is performed by using the apparatus shown in FIG. 36;

FIG. 39 presents the microscopic picture of FIG. 38 as it is magnified;

FIG. 40 presents a microscopic picture of a fluid that has been obtained one minute after the experimental testing was performed by using the apparatus shown in FIG. 36;

FIG. 41 presents a microscopic picture of a fluid that has been obtained three minutes after the experimental testing was performed by using the apparatus shown in FIG. 36;

FIG. 42 presents a microscopic picture of a fluid that has been obtained five minutes after the experimental testing was performed by using the apparatus shown in FIG. 36;

FIG. 43 shows the apparatus for mixing and/or crushing substances into fine particles in accordance with one embodiment of the present invention, in which FIG. 43(a) represents the front view and FIG. 43(b) represents the side elevation;

FIG. 44 is a schematic diagram illustrating how two units of the apparatus of FIG. 43(a) are connected with each other;

FIG. 45(a) represents a cross section of the inlet and outlet sides of the apparatus of FIG. 43(a) with some non-critical parts being not shown, and FIG. 45(b) represents a cross section of the central portion of the apparatus of FIG. 43(a) with some non-critical parts being not shown;

FIG. 46(a) is a plan view illustrating how the frame members are mounted inside the apparatus shown in FIG. 43(a), and FIG. 46(b) is a plan view illustrating how the structural members are also mounted inside the apparatus;

FIG. 47(a) is a front elevation illustrating how the fluid flow paths are formed by the frame members inside the apparatus shown in FIG. 43(a), and FIG. 47(b) is a front elevation illustrating how the fluid flow paths are also formed by the structural members inside the apparatus;

FIG. 48(a) is a plan view showing a first plate member employed in the apparatus shown in FIG. 43(a), FIG. 48(b) represents a cross section of the first plate member taken along the line F-F, and FIG. 48(c) represents a perspective view of the first plate member;

FIG. 49(a) is a plan view showing a second plate member employed in the apparatus shown in FIG. 43(a), FIG. 49(b) represents a cross section of the second plate member taken along the line F-F, and FIG. 49(c) represents a perspective view of the second plate member;

FIG. 50(a) is a plan view illustrating how the frame members are placed one over the other inside the apparatus shown in FIG. 43(a), FIG. 50(b) represents a cross section of the frame members taken along the line G-G and FIG. 50(c) represents a perspective view of the frame members;

FIG. 51 illustrates how a fluid flows through the apparatus shown in FIG. 43(a) so that the fluid can be mixed and/or crushed into fine particles in accordance with different embodiments of the present invention, in which FIG. 51(a) represents a cross section according to the first embodiment, and FIG. 51(b) represents a-cross section according to the second embodiment;

FIG. 52 is a front perspective view of the apparatus for mixing and/or crushing substances into fine particles in accordance with one embodiment of the present invention;

FIG. 53 is a diagram that illustrates how the shape of a cell having one front side open is defined so that the cell can be formed on the surface of a structure in order to provide a fluid flow path in accordance with the embodiment of FIG. 52;

FIG. 54(a) is a front elevation illustrating the cylindrical casing in the apparatus shown in FIG. 52, FIG. 54(b) is an exploded view of the components that make up the individual fluid conducting unit in the apparatus of FIG. 52, and FIG. 54(c) is a front elevation illustrating how the individual fluid conducting units are coupled together in the apparatus of FIG. 52;

FIG. 55 is a perspective view illustrating how a fluid flows through the apparatus of FIG. 52 so that the fluid can be mixed and/or crushed into fine particles;

FIG. 56 is a front elevation illustrating how soybeans are crushed into very fine particles in the apparatus of FIG. 52 in accordance with one embodiment of the present invention;

FIG. 57 is a block diagram showing the methods for mixing and/or crushing substances into fine particles in accordance with different embodiments of the present invention that may be employed in the apparatus of the present invention, in which FIG. 57(a) is a block diagram of the method according to a first embodiment, FIG. 57(b) is a block diagram of the method according to a second embodiment, and FIG. 57(c) is a block diagram of the method according to a third embodiment;

FIG. 58 is a block diagram showing the continuous ultracritical process of crushing plastic wastes into fine particles that may take place in the apparatus of the present invention in accordance with one embodiment of the present invention; and

FIG. 59 is a schematic diagram illustrating the operation of a typical conventional stationary mixing apparatus, in which FIG. 59(a) represents a cross section of the apparatus, FIG. 59(b) represents a perspective view of a larger-diameter round plate provided in the apparatus, and FIG. 59(c) represents a perspective view of a smaller-diameter round plate provided in the apparatus.

### BEST MODES OF EMBODYING THE INVENTION

Now, the present invention will be described in further detail with reference to several preferred embodiments of the present invention by referring to the accompanying drawings. It should be understood that the arrangements, shapes and positional relationships of the machine components, elements and parts that will be described below by referring to the accompanying drawings are only given in general terms that help understand the concept of the present invention. It should also be understood that the specific values and compositions (material types) of the machine components, elements and parts are only given by way of examples. It should be appreciated, therefore, that the present invention is not limited to any of the embodiments that will be described below, and should only be understood from the technical scope of the invention as defined in the appended claims and may be modified in various ways and manners.

### Embodiment 1

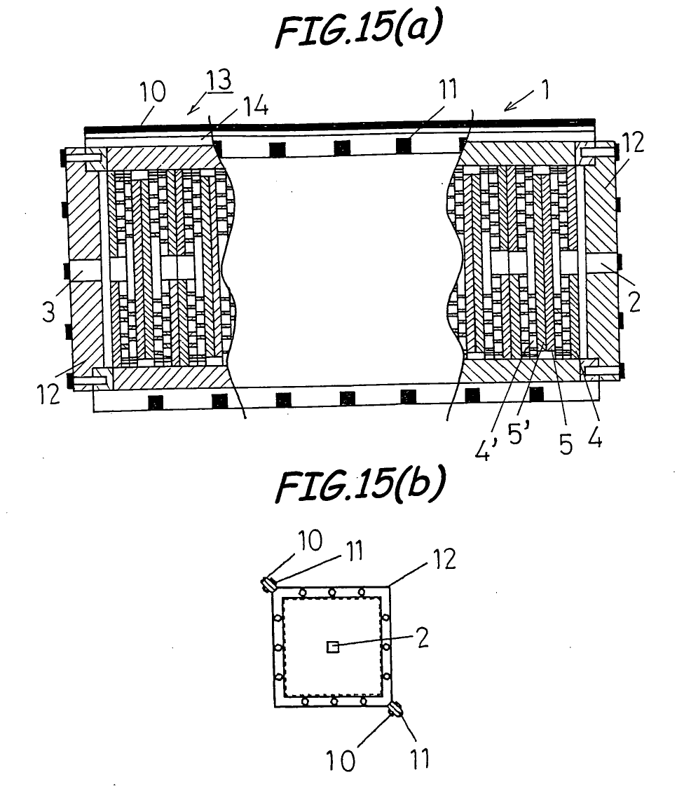

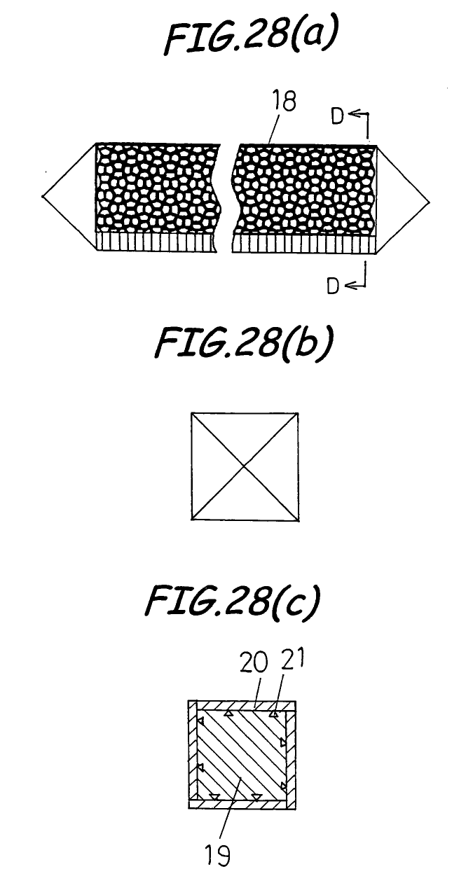

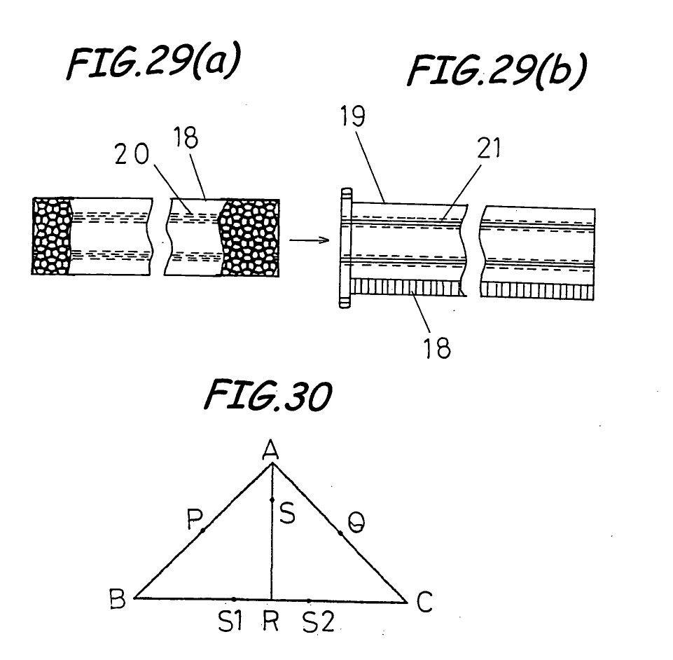

Referring first to FIGS. 15(a) through FIG. 29(b), the first embodiment of the present invention is described.

FIG. 15(a) is a schematic diagram that represents the cross section of an apparatus for mixing and/or crushing substances into fine particles. As shown in FIG. 15(a), the apparatus comprises a cylindrical casing 1 that has a hollow portion therein, and has an inlet 2 on one end and an outlet 3 on the opposite end. The cylindrical casing 1 further includes a fluid flow path that is formed by a first structural member and a second structural member.

In the following description, the apparatus for mixing and/or crushing substances into fine particles may be referred to simply as the apparatus for the convenience of simplicity, but it should be understood that this means the apparatus for mixing and/or crushing substances into fine particles, except otherwise specified.

The first and second structural members includes a plurality of first plate members 4 and a plurality of second plate members 5, respectively, which are placed one over another in the direction perpendicular to the axial direction of the cylindrical casing 1, thereby forming the fluid flow path.

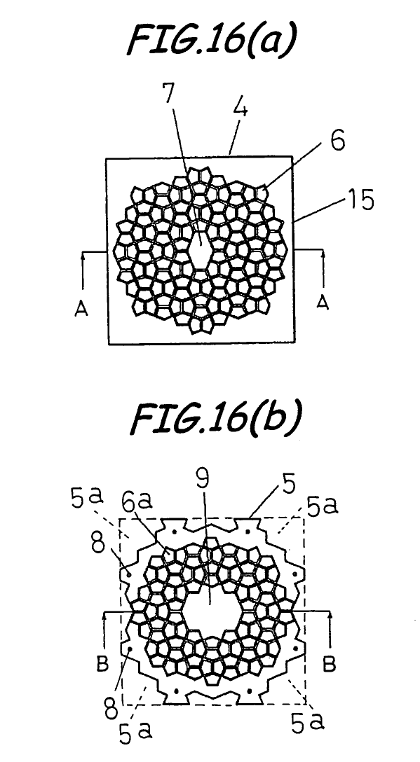

As shown in FIG. 16(a), each of the first plate members 4 has its outer circumferential shape that matches the inner circumferential shape of the hollow portion inside the cylindrical casing 1, and may be mounted inside the cylindrical casing 1 in such a way that it can make intimate contact with the inside of the hollow portion. Each first plate member 4 has a plurality of pentagonal (five-sided) cells 6 thereon that are arranged like a honeycomb. Each of the cells 6 has its front side open, and the cells 6 are configured to provide a center hole 7 though the configuration.

Each of the second plate members 5 has its outer circumferential shape such that the second plate member 5 can be mounted inside the hollow portion inside the cylindrical casing 1, by allowing a gap to be formed between the outer circumference of the second plate member 5 and the inner circumference of the hollow portion inside the cylindrical casing 1. Each second plate member 5 has a plurality of pentagonal (five-sided) cells 6a thereon that are arranged like a honeycomb. Each of the cells 6a has its front side open, and the cells 6a are configured to provide a center recess 9 thereon.

The apparatus shown in FIG. 15(a) includes the cylindrical casing 1 which may have the appearance of a rectangular shape or cylindrical shape (FIG. 22), and has flanges 10, 10 as shown in FIG. 15(b). Each of the flanges 10, 10 is located at each corresponding one of the corners on the diagonal line across the cylindrical casing 1, extending outwardly. Those flanges 10, 10 are provided with bolts 11, through which the casing 1 can be assembled or disassembled easily.

In the embodiment shown in FIGS. 15(a) and (b), the cylindrical casing 1 has the hollow portion inside it, whose inner circumference has a substantially square cross section. Similarly, the first and second plate members 4, 5 that are mounted in the casing 1 also have a substantially square cross section as shown in FIGS. 16(a) and (b).

The apparatus for mixing and/or crushing substances into fine particles has a cover 12 on each of the opposite ends thereof. Each of the covers 12 has a square cone shape, and may be mounted removably on each of the opposite ends. The cover 12 on the inlet side has an inlet opening 2 at the center, through which an object being processed, such as being mixed or crushed, may be fed into the casing 1, whereas the cover 12 on the outlet side has an outlet opening 3 at the center, through which the substances that have been processed, such as being mixed or crushed, may be discharged. The inlet opening 2 and outlet opening 3 may have any geometric shape.

In accordance with the present invention, the component members that constitute the apparatus, such as the cylindrical casing 1, first and second plate members 4, 5 and others, may be made of any of the metal or non-metal materials. Such materials may include carbons, composite metal materials such as combinations of carbon and copper, carbon and aluminum, carbon and magnesium, carbon and tungsten, carbon and titanium oxide, mineral materials such as ceramics, tourmaline and the like, and natural or synthetic resins.

As shown in FIG. 15(a), the first and second structural members that may be installed inside the hollow portion of the cylindrical casing 1 for forming a fluid flow path includes first plate members 4 and second plate members 5, respectively, which are placed one over another in the direction perpendicular to the axial direction of the cylindrical casing 1. It should be noted that the first plate members 4 and second plate members 5 may be installed inside the hollow portion by placing those members one over another in the horizontal direction from the right toward left sides in FIG. 15(a).

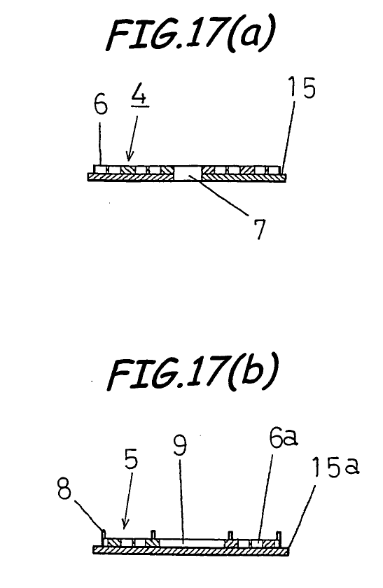

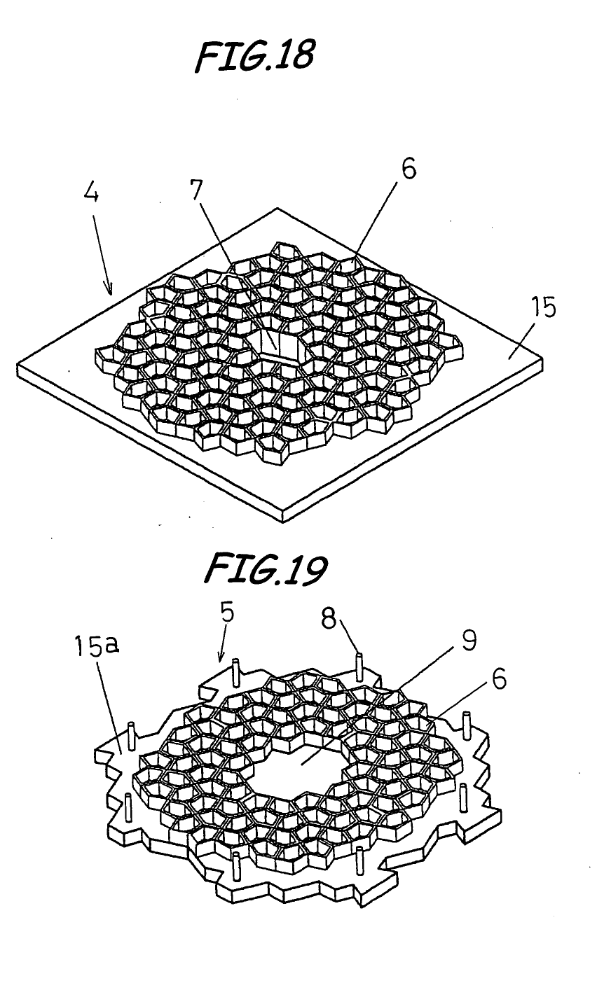

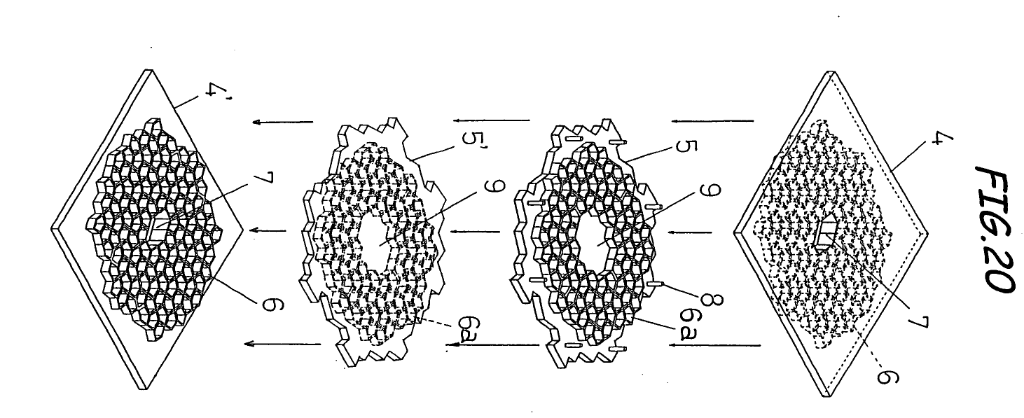

FIG. 16(a) is an enlarged plan view of the first plate member 4, FIG. 16(b) is an enlarged plan view of the second plate member 5, and FIGS. 17(a) and (b) represent a cross section taken along the line A-A in FIG. 16(a) and a cross section taken along the line B-B in FIG. 16(b), respectively. FIG. 18 represents a perspective view of the first plate member 4, and FIG. 19 represents a perspective view of the second plate member 5.

It may be seen from FIG. 16(a) through FIG. 19 that the first plate member 4 includes a square base plate 15 that is slightly smaller than the section 14 located inside the cylindrical casing 1 where the plate member is mounted. The base plate 15 has a plurality of pentagonal cells 6 each having its front side open, and has a through hole 7 at the center that has a hexagonal shape having four longer sides and two shorter sides that is formed like the shape of a group of four cells 6. Outwardly of the center hole 7, groups of four pentagonal cells 6 each having its front side are arranged in the succeeding manner.

The second plate member 5 includes a square base plate 15a (FIG. 19) having four sides, each of which has cutout portions 5a at certain areas with two projecting sides. The base plate 15 has a recess 9 at the center that is formed like the shape of a group of fourteen (14) pentagonal cells 6a each having its front side open. Like the first plate member 4, outwardly of the center recess 9, groups of fourteen (14) pentagonal cells 6a each having its front side are arranged in the succeeding manner. The second plate member 5 has stability pins 8 (FIG. 19), each of which is provided on each of the projecting sides so that it can ensure the stability of the first and second plate members when they are placed one over the other,

The second plate member 5 may include a base plate 15a that is formed to have cutouts on the four side corners 5a. The second plate member plate 5 may have any shape having the outer diameter that matches that of the first plate member 4. What is important is that there should be a space that allows a fluid to flow from one plate member to another adjacent plate member located downstream of the one plate member.

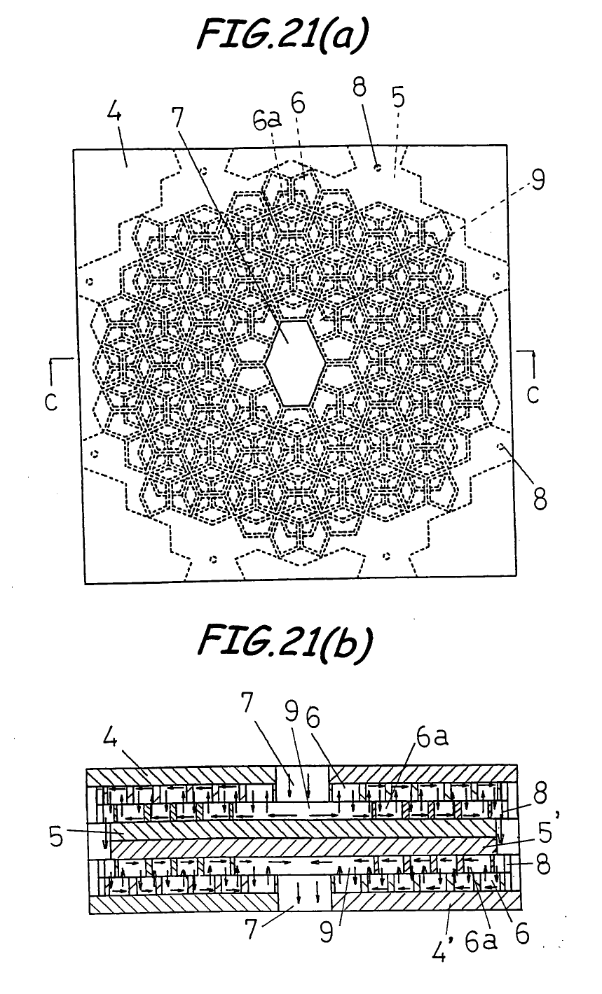

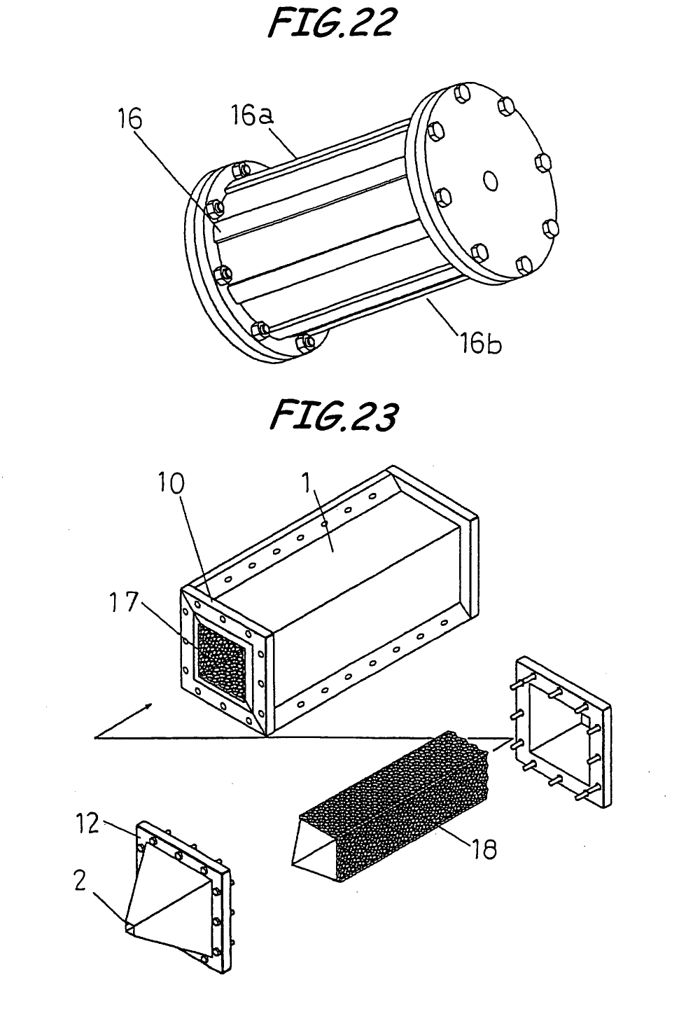

FIG. 20 represents a perspective view illustrating how the first plate member 4 and second plate member 5 are placed one over the other to form a single fluid conducting unit. FIG. 21(a) represents a plan view of the unit that includes the first and second plate members placed one over the other, and FIG. 21(b) represents a cross section taken along the line C-C in FIG. 21(a).

The following describes how the first plate member 4 and second plate member 5, part of which is shown in FIG. 15(a), may be placed one over the other, by referring to FIG. 20.

Initially, the first plate member 4 and second plate member 5 may be placed one over the other so that the center hole 7 on the first plate member 4 can be aligned with the center recess 9 on the second plate member 5, with each of the pentagonal cells 6 having its front side open on the first plate member 4 being placed to face opposite each of the pentagonal cells 6a having its front side open on the second plate member 5 to communicate with each other through the respective open front sides and with each cell on the first plate member 4 being placed in alternate positions with regard to each corresponding cell on the second plate member 5. For example, the first and second plate members 4 and 5 may be placed by making the intimate contact with each other so that each cell 6 on the first plate member 4 and each cell 6a on the second plate member 5 that is placed opposite each corresponding cell 6 on the first plate member 4 may be oriented reversely by turning one relative to the other by an angle of 90 degrees. Thus, when the first and second plate members are placed so that the cells 6 on the first plate member 4 may be placed to face opposite the corresponding cells 6a on the second plate member 5, each of the cells on one plate member may communicate with at least one or more of the cells on the other plate member. Then, another second plate member 5′ may be placed back-to-back on the second plate member 5. Then the another plate member 5′ may be placed on another first plate member 4′ that is also placed back-to-back on the first plate member 4 so that the center hole 7 on the another first plate member 4 can be aligned with the center recess 9 on the another second plate member 5′ and so that each of the pentagonal cells 6 having its front side open on the another first plate member 4′ is placed to face opposite each of the pentagonal cells 6 having its front side open on the another second plate member 5′ through the respective open front sides, with each cell 6 on the another first plate member 4′ being placed in alternate positions with regard to each corresponding cell 6 on the another second plate member 5′. Thus, the another second plate member 5′ and another first plate member 4′ are placed one over the other so that each of the cells on one plate member may communicate with at least one or more of cells on the other plate member.

It may be appreciated that the fluid conducting unit may thus include the first plate member 4, second plate member 5, another second plate member 5′, and another first plate member 4′ that are placed one over another. FIG. 15(a) shows that several units are installed inside the cylindrical casing 1, but a single unit may also be installed.

It may be understood from the foregoing description that the first plate member 4 and second plate member 5 are placed one over the other such that the two plate members can contact each other intimately, with the pentagonal cells 6 on the first plate member 4 being placed opposite the pentagonal cells 6a on the second plate member 5 and with each of cells 6 being displaced relative to each cells 6a by turning one relative to the other by an angle of 90 degrees. Thus, the cells 6 or 6a are arranged intimately like a honeycomb with no gap between any adjacent cells 6 or 6a as shown in FIG. 16(a) or (b), and the space on the front side of the cells 6 or 6a is divided into three parts as shown in FIG. 21(a).

When a fluid is introduced under the applied pressure into the apparatus 13 so that the fluid can be mixed and/or crushed into fine particles, the fluid may first pass through the center through hole 7 on the first plate member 4, and may then hit the recess 9 on the second plate member 5, as shown in FIG. 21(b), flowing through the front space divided into the three parts into the upper and lower pentagonal cells 6, where the fluid hits the cells repeatedly, dispersing radially toward the outside. The fluid that has dispersed or diffused up to the outer circumferential side goes to the base plate 15 in the first plate member 4 where it hits the base plate 15, flowing through the front space divided into the three parts into the upper and lower pentagonal cells 6, where the fluid hits the cells repeatedly, flowing toward the center. The above process, during which the fluid hits cells where its flow is reversed, is repeated throughout the cylindrical casing 1, and the fluid may be finally mixed and/or crushed into fine particles.

The components that constitute the apparatus 13 described above, such as the cylindrical casing 1, first plate member 4, second plate member 5 and the like, may be made of any of the materials that may include carbons, metal-carbon combination materials such as carbon and copper, carbon and aluminum, carbon and magnesium, carbon and tungsten, and carbon and titanium oxide, and mineral materials such as ceramics, tourmaline and the like. The components that are made of any of the materials mentioned above may also provide the excellent catalyst action.

FIG. 22 is a perspective view illustrating the external shape of the cylindrical casing 1 as one component of the apparatus 13, which is formed like a cylindrical shape in this case.

In the embodiment shown in FIG. 22, the cylindrical casing 1 has its outer circumference that is divided into eight sections along the axial direction of the casing 1, and magnets 16 are disposed on the eight sections such that a magnet 16 on one section may provide N polarity 16a, for example, and a magnet 16 on the section opposite the one section may provide S polarity, for example. Those magnets 16 may attract a fluid magnetically when it is introduced under the applied pressure into the cylindrical casing 13 on the apparatus 13. Thus, the molecules in the fluid may be subdivided more finely, and mixed and/or crushed into fine particles more efficiently under the magnetic action of the magnets 16.

It should be noted that the cylindrical casing in the apparatus may have other external shapes. For example, the cylindrical casing 1 may include a flange that extends from the center along the horizontal direction, and may be divisible into two parts along the horizontal direction.

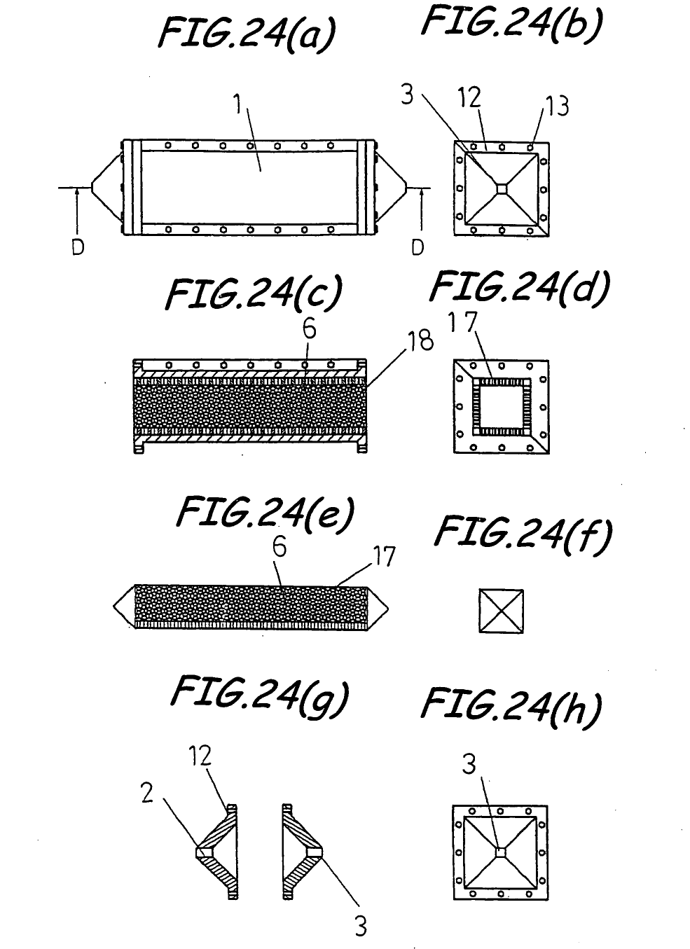

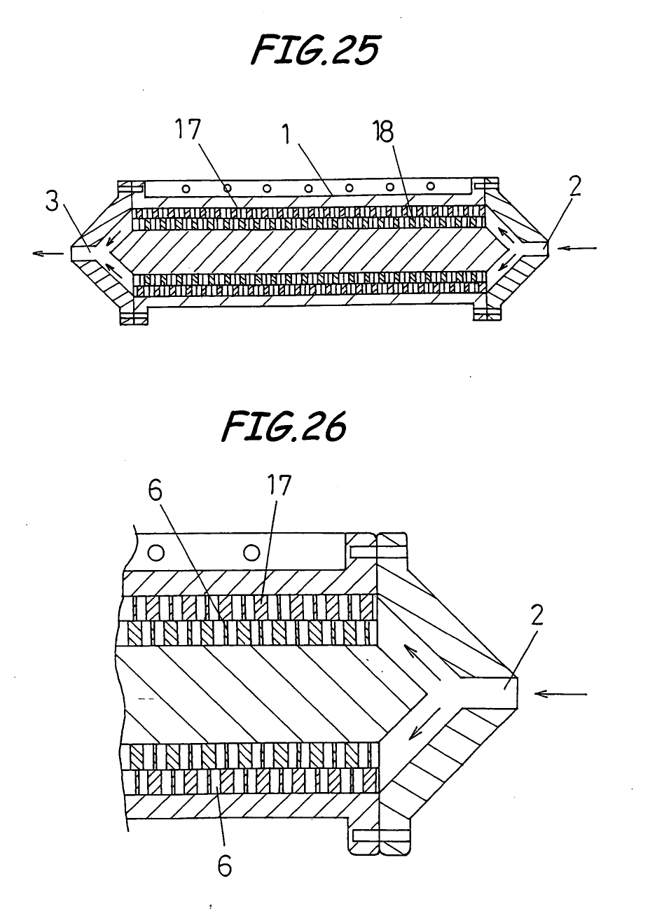

FIGS. 23 through 26 represent other possible embodiments. Specifically, FIG. 23 represents the exploded perspective view, FIGS. 24(a) through (h) represent the exploded view, and FIGS. 25 and 26 represent the sectional view and partly enlarged sectional view illustrating the flow of the fluid, respectively.

In those embodiments, the cylindrical casing 1 includes a plate member 17 having the inner circumferential wall formed like a square having four sides, and each of the four walls has a plurality of pentagonal cells 6 thereon, each having its front side open, that are arranged like a honeycomb. The cylindrical casing 1 further includes a structural member that may be fitted inside the cylindrical casing 1. The structural member is formed by a plate member 18 having the outer circumferential wall formed like a square having four sides, and each of the four walls has a plurality of pentagonal cells 6, each having its front side open, that are arranged like a honeycomb. With the plate member 18 being fitted inside the cylindrical casing 1, the pentagonal cells 6 on the plate member 17 may face opposite the pentagonal cells 6 on the plate member 18, with the former cells 6 being placed in alternate positions with regard to the latter cells. The fluid flow path may thus be formed through the open front sides of the cells facing opposite each other. The cylindrical casing further includes a flange on each of the opposite ends, to which a cover 12 may be mounted.

Substances being processed, such as being mixed and/or being crushed into fine particle crushing, take the form of a fluid, which may be introduced under the applied pressure into the cylindrical casing 1 through the inlet 2 on the cover 12. Then, the fluid may flow into the space formed like a square pyramid in the cover 12, through which the fluid may go toward the location where the plate members 17, 18 are placed one over the other, with the open front sides of the pentagonal cells 6 on one plate member facing opposite the open front side of the cells 6 on the other plate. Going through the fluid flow path formed by the pentagonal cells 6, the flow may collide with the lateral side of each of the pentagonal cells 6, and may then reverse its flow. As the fluid flows though its flow path, it may hit the base, top and lateral sides of each pentagonal cell, and may then reverse its flow, Then, the fluid may be divided into several flows, which may collide with each other, and may then reverse the respective flows when going further through each of the pentagonal cells 6. This process may be repeated through the total flow path until the fluid reaches the outlet 3.

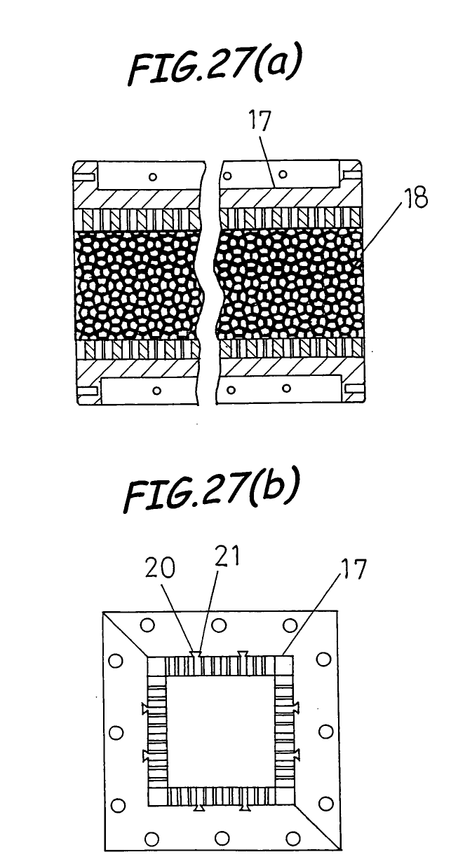

FIG. 27(a) through FIG. 29(b) represent other embodiments of the present invention, wherein a collection of continuous plate members 17 each having a plurality of pentagonal cells 6, each having its front side open, that are arranged like a honeycomb may be mounted on the four inner sides of the cylindrical casing 1, and a collection of continuous plate members 18 may be mounted on the respective sides on the base 19.

The cylindrical casing 1 and base 19 have a number of substantially triangular recesses 20 formed in appropriate locations on the four faces thereof, respectively, and the plate member 17 and plate member 18 have a number of substantially triangular projections 21 formed on the sides opposite sides on which the pentagonal cells 6 are provided so that the projections 21 can engage the corresponding recesses 20. Those recesses 20 and projections 21 make it easy to replace the plate members 17 and plate members 18 that are made of different materials.

### Embodiment 2

In the preceding embodiment, each cell 6 having its front side open has the pentagonal shape. It should be appreciated that the cell may have any other shape other than the pentagonal shape.

FIG. 30 represents other possible shapes that can be defined for the cells, each having its front side open, that are provided on the plate members.

As shown in FIG. 30, a right-angled isosceles triangle ΔABC having a right-angle vortex A is given, in which middle points P and Q on the hypotenuse are designated as absolute points. Then, an imaginary point S on the perpendicular from the vortex A to the base B-C may be set on any location other than the vortex A and the midpoint R on the base. Different lines that can be drawn to the imaginary point S, starting at the midpoints P, Q on the hypotenuse may be turned clockwise about the midpoint P as the origin, and anticlockwise about the midpoint Q as the origin. Then, the lines may intersect on point S1, S2 on the base B-C. The shape that is surrounded by the points P-S-Q-S2-S1-P may be used as the shape for each of the cells having its front side open.

It may be appreciated that the imaginary point S may be set on any location on the perpendicular A-R, and a leg on the base may be formed by turning the line segment connecting between the midpoints P and Q on the hypotenuse and the origin clockwise and anticlockwise.

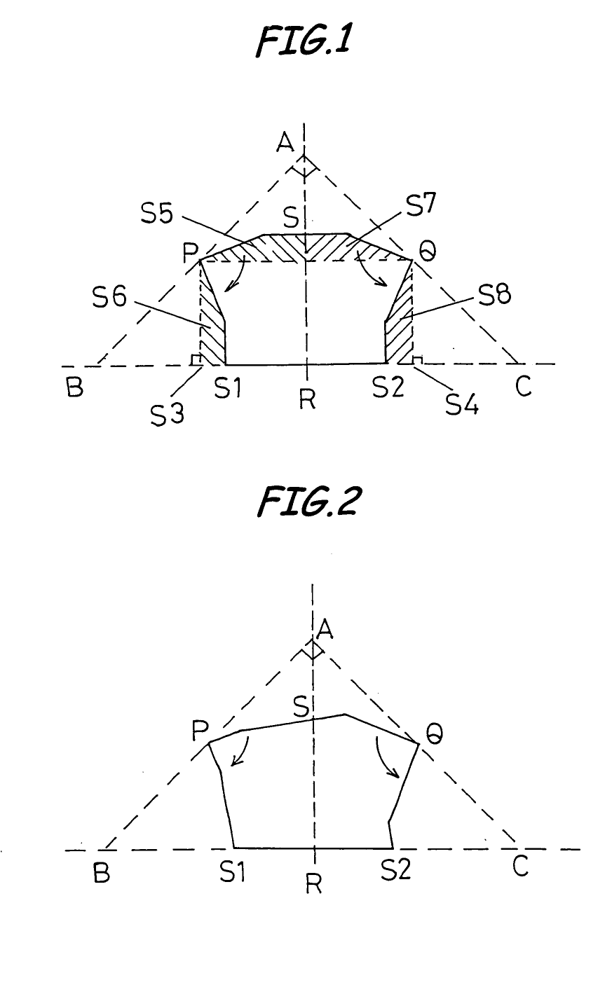

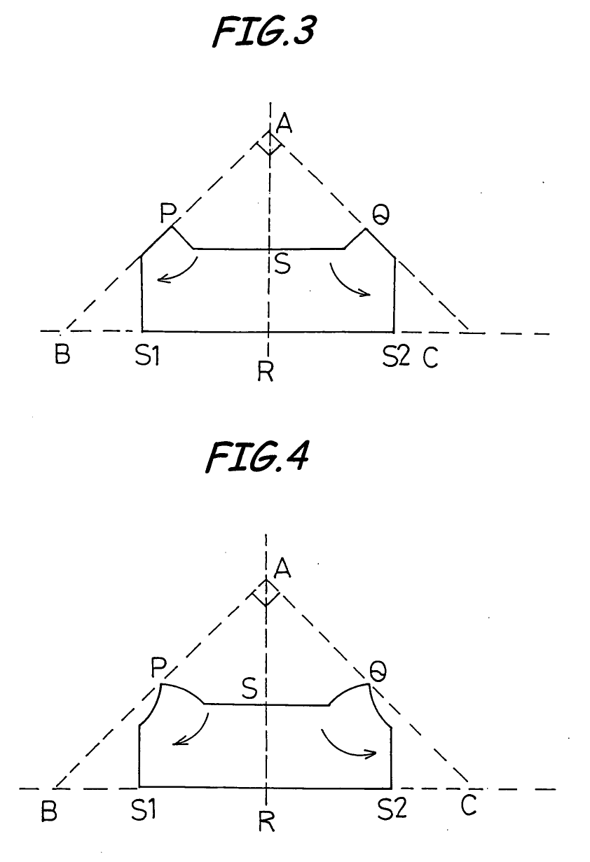

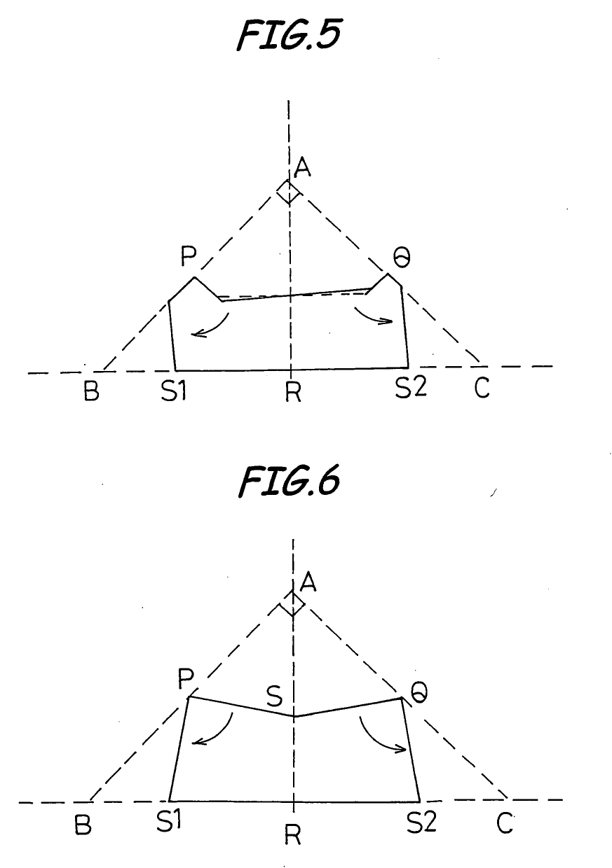

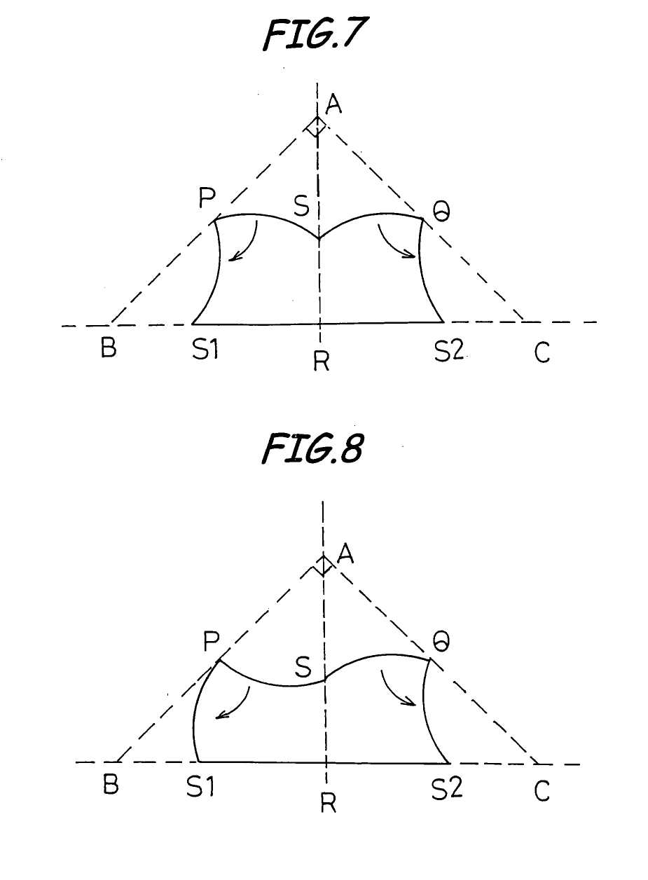

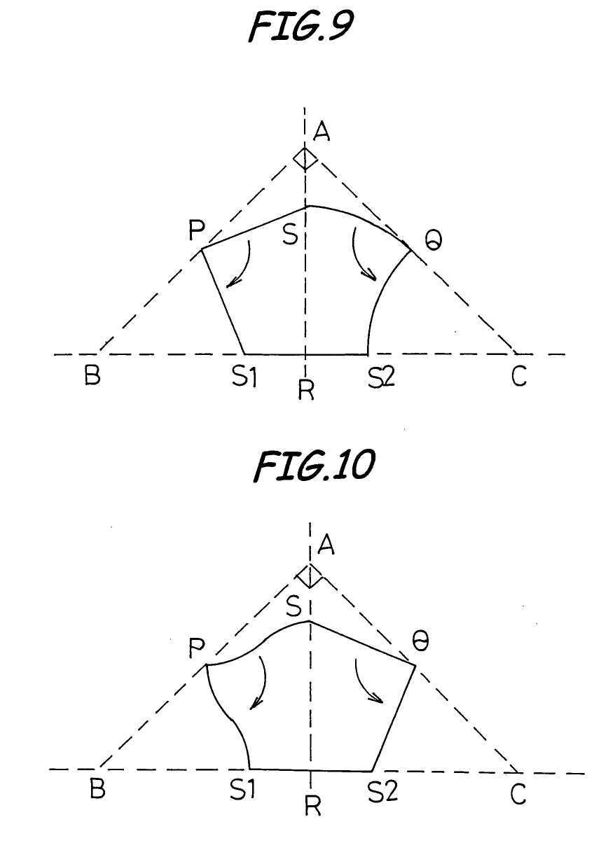

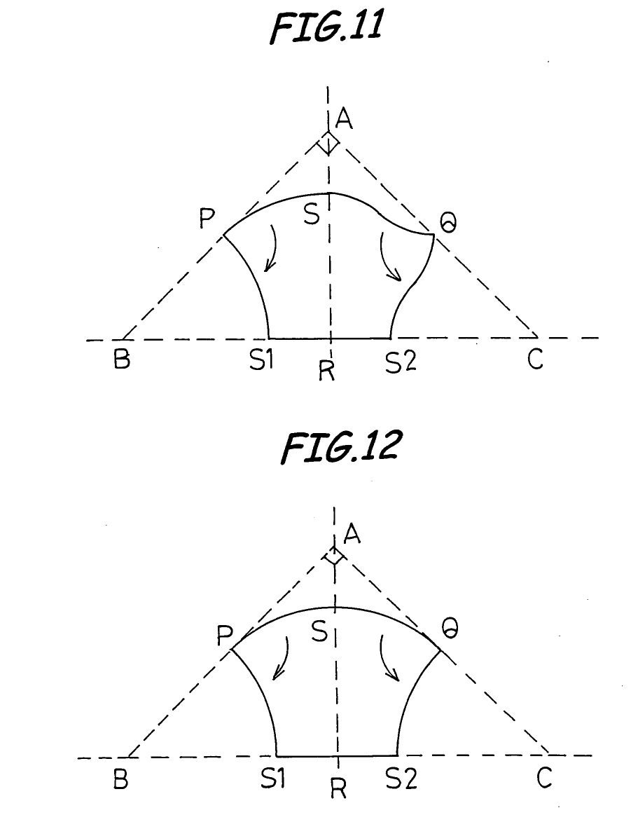

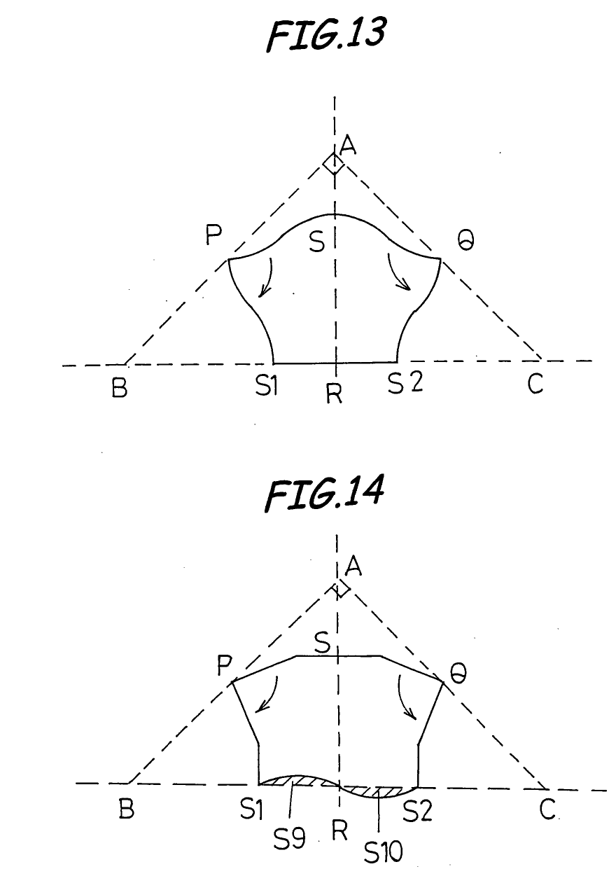

The cell may have other different shapes than the shape described above, as shown in FIGS. 1 through 14. Even when the cells have any of the shapes shown, they can be placed on the entire plate member like the honeycomb, without producing any gaps between any two adjacent cells.

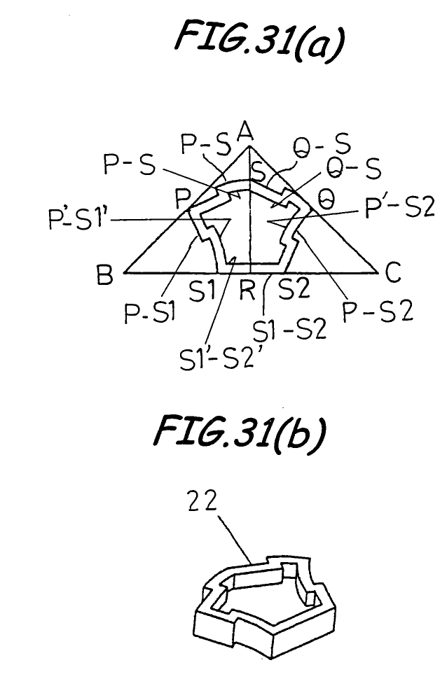

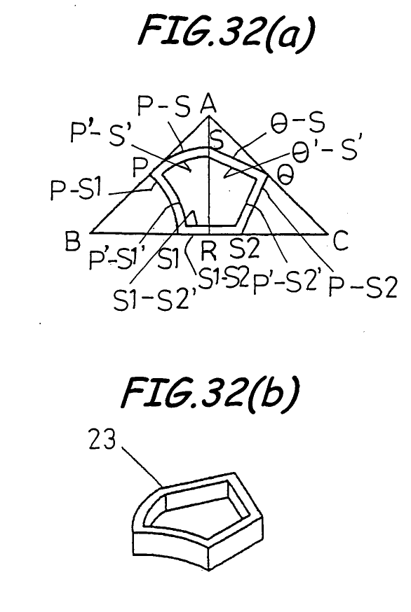

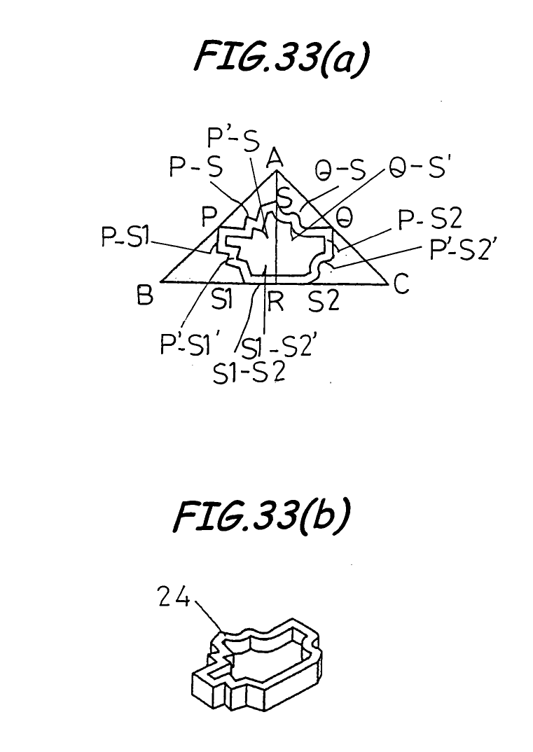

FIGS. 31(a) through 33(b) represent the specific examples of the cell shapes described by referring to FIG. 30, in which FIG. 31(b), FIG. 32(b), and FIG. 33(b) represent the perspective view of the cell formed in accordance with the steps shown in FIG. 31(a), FIG. 32(a), and FIG. 33(a), respectively.

Specifically, it is shown in FIG. 31(a) that when an imaginary right-angled isosceles triangle ΔABC having the right-angle vortex A, two hypotenuses A-B and A-C, and the base B-C is given, midpoints P, Q, R may be set on the hypotenuses A-B and A-C, and the base B-C, and point S may be set on any location other than points A, R on a line segment A-R connecting between the vortex A and midpoint R. The line segment P-S connecting between the vortex A and midpoint S is a combination line that includes an arc line from the midpoint on P-S toward point S, an arc line extending as straight line toward the midpoint and a straight line. The line segment connecting between midpoint Q and point S is a combination line that includes a straight line from the midpoint on P-S toward point S, a straight line extending as arc line toward midpoint Q, and an arc line. Then, the line segment P-S may be turned clockwise about midpoint P as the origin, and the line segment Q-S may be turned anticlockwise about midpoint Q as the origin, and the points of contact on the base B-C that result may be designated as S1 and S2. Connecting points P, S, Q, S2, S1, P may then produce an external shape that is asymmetrical. The portion that is sandwiched between the shape P-S-Q-S2-S1-P and the shape surrounded by segment P′-S′, line segment Q′-S′, line segment P′-S1′, line segment S1′-S2′, and line segment Q′-S2′ that is similar to but smaller than the above shape may be used as the wall of a cell 22.

It may be appreciated that the base S1-S2 may be any other line type, not the straight line, such as the segment S1′-R that may be a sine line, and S2-R that may be a straight line. It should be understood, however, that it is desirable that the shape of the portion connected by the line segment P-S-Q-S2-S1-P should have the area that is equal to one half the area of the imaginary right-angled isosceles triangle.

In FIG. 32(a), when an imaginary right-angled isosceles triangle ΔABC having the right-angle vortex A, two hypotenuses A-B and A-C, and the base B-C is given, midpoints P, Q, R may be set on the hypotenuses A-B and A-C, and the base B-C, and point S may be set on any location other than points A, R on a line segment A-R connecting between the vortex A and midpoint R. The line segment P-S connecting between midpoints P and midpoint S and point S is designated as a convex arc line, and the line Q-S is designated as a straight line. Then, the line P-S may be turned clockwise about the midpoint P as the origin, and the Q-S may be turned anticlockwise about the midpoint Q as the origin. The points of contact on the base B-C that may result are designated as S1 and S2. Connecting points P, S, Q, S2, S1, P may produce an external shape that is asymmetric. The portion that is sandwiched between the shape P-S-Q-S2-S1-P and the shape surrounded by segment P′-S′, line segment Q′-S′, line segment P′-S1′, line segment S1′-S2′, and line segment Q′-S2′ that is similar to but smaller than the above shape may be used as the wall of a cell 23, as shown in FIG. 32(b).

In FIG. 33(a), when an imaginary right-angled isosceles triangle ΔABC having the right-angle vortex A, two hypotenuses A-B and A-C, and the base B-C is given, midpoints P, Q, R may be set on the hypotenuses A-B and A-C, and the base B-C, and point S may be set on any location other than points A, R on a line segment A-R connecting between the vortex A and midpoint R. The line segment P-S connecting between midpoints P and midpoint S and point S is designated as a flexible broken line, and the line Q-S is designated as the corner line R of the flexible broken line. Then, the line P-S may be turned clockwise about the midpoint P as the origin, and the Q-S may be turned anticlockwise about the midpoint Q as the origin. The points of contact on the base B-C that may result are designated as S1 and S2. Connecting points P, S, Q, S2, S1, P may produce an external shape that is asymmetric. The portion that is sandwiched between the shape P-S-Q-S2-S1-P and the shape surrounded by segment P′-S′, line segment Q′-S′, line segment P′-S1′, line segment S1′-S2′, and line segment Q′-S2′ that is similar to but smaller than the above shape may be used as the wall of a cell 23, as shown in FIG. 33(b).

FIGS. 23(a) and (b) represent how the cells 22 having the shapes defined above are arranged like the honeycomb on the surface of the plate member. The top and bottom are formed by a wall 25 that is as high as the inner circumferential wall of the cylindrical casing 1 that forms part of the apparatus.

FIGS. 85(a) and (b) represent how the cells 22 having the shapes defined above are arranged like the honeycomb on the front and rear sides of the plate member. When any two adjacent plate members are placed to adjoin each other, the cells 22 on the front side of one plate member and the cells on the rear side of the other plate member are arranged such that the cells on one plate member are placed in alternate positions with respect to the cells on the other plate member, with the cells on both plate members being displaced by an angle of 180 degrees. In this case, the top and bottom are also formed by a wall 25 that is as high as the inner circumferential wall of the cylindrical casing 1 that forms part of the apparatus according to the present invention.

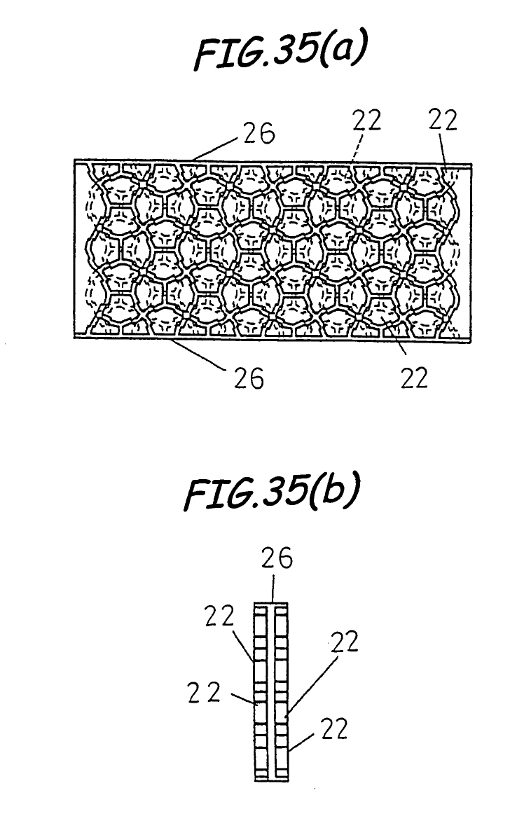

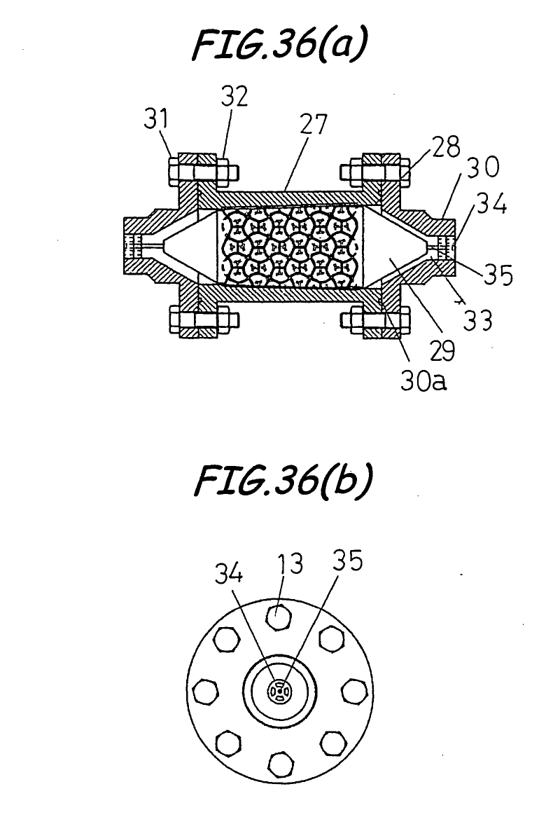

FIGS. 36(a) and (b) represent one specific example of the apparatus in accordance with the present invention, wherein the apparatus includes a plurality of plate members shown in FIGS. 35(a) and (b) that are placed one over another inside the cylindrical casing 1 in the axial direction of the cylindrical casing 1.

The cylindrical casing 1 has the appearance of a cylindrical casing 27 having a flange extending from each of the opposite ends. The cylindrical casing 27 has an internal space 28 therein, which is formed like a frustoconical shape, and includes a plurality of plate members shown in FIGS. 35(a) and (b), which are arranged regularly on the entire inner circumferential wall of the internal space 28. A member 29 that may be fitted tightly into the internal space 28 has the conical shape.

Each of flanges on the opposite ends of the cylindrical casing 27 has an O-ring 30a that is provided as a seal for the fluid. The cover 30 may be mounted on each of the opposite ends of the cylindrical casing 27 by tightening bolt 31 and nuts 32, so that the cover 30 can keep the internal space 28 airtight. The cover 30 has an internal space 33 therein, which has a conical shape that is slightly larger than the conical shape on the opposite ends of the member 29. Substances being processed, such as being mixed and/or being crushed, take the form of a fluid that may be fed into the cylindrical casing 27 through its inlet 34. Inside the inlet 34, there is a spiral fluid mechanism that is fastened by a bolt 35.

For the apparatus shown in FIG. 36(a), the inner space 28 has the frustoconical shape, and the member 29 fitted into the inner space 28 also has the frustoconical shape. When the fluid of substances being mixed and/or crushed are fed into the cylindrical casing 1 through its inlet 34, the fluid may flow through the paths through which each of the cells 22 on one of any two adjacent plate members placed one over the other in the axial direction of the cylindrical casing 1 can communicate with at least one or more cells on the other plate member opposite the one plate member, and can have the mixing and/or crushing process during which fine particles can be generated or formed.

The apparatus shown in FIG. 36(a) may be used as a mixer, crusher or device for providing fine particles in the spherical forms. It may also be used as a device for generating a critical fluid or ultracritical fluid by causing the substances being processed to react when the pressure and temperature are placed under the ultracritical condition above the gas-liquid critical point.

More specifically, the apparatus shown in FIG. 36(a) may be constructed by using the structural members that are capable of resisting the critical and ultracritical temperature conditions under which the substances are being mixed and/or crushed into fine particles. Thus, the apparatus shown in FIG. 36(a) may be used as a reactor vessel in which the process of mixing and/or crushing substances into fine particles can be performed under the critical and ultracritical processing conditions.

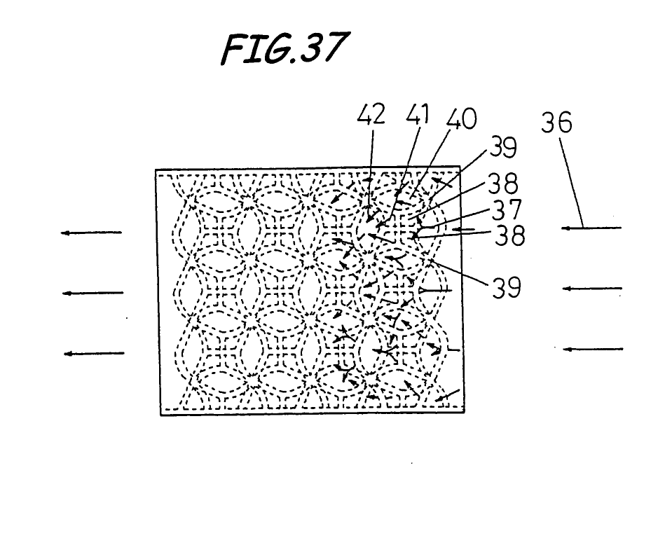

FIG. 37 is a diagram illustrating how the substances being processed, such as being mixed and/or crushed, can flow through the fluid flow path within the cylindrical casing 27 in accordance with the embodiment shown in FIG. 36(a). There are two plate members that are placed adjacently to each other such that the open front sides of the cells 22 on one plate member face opposite the open front sides of the cells 22 on the other plate member, with the cells 22 on the one plate member being rotated through an angle of 180 degrees with regard to the cells 22 on the other plate member. Those plate members may be installed within the cylindrical casing 27.

When a fluid of substances being processed is fed under the applied pressure into the cylindrical casing 27 through its inlet 34, the fluid flows through the continuous sequence of divisional sections, flowing first into the first divisional section 37 having the area and volume larger than the other divisional sections, and then flowing into the next following divisional section 38. The fluid 36 flowing through the first divisional section 36 is blocked by the walls 39 formed by midpoints P and P′ and midpoints Q and Q′ as described earlier and that overlap each other vertically, hitting against the walls forming the cells 22 and going over the walls into the next following divisional section 38. The divisional section 38 has the area and volume that are smaller than the area and volume of the divisional section 37 by about 90%, and thus may include the number of divisions that doubles that of the divisional section 37, The fluid can flow faster when it goes through the divisional section 38.

Then, the fluid 38 goes to the next following divisional section 40 that has the area and volume that are increased by 2.15 as compared with the divisional section 38, where the fluid flows more slowly. Going further into the next following divisional section 41, the fluid can flow faster again, going into the next following divisional section 42 where the fluid flows more slowly.

It may be understood from the above description that the fluid 36 can flow through the continuous sequence of divisional sections having the different areas and volumes, and when the fluid 36 passes through the smaller divisional section, it can flow at a faster rate, and can be placed under the embracing pressure that provides the actions of the increased compression and coagulation against the fluid, When the fluid 36 passes through the larger divisional section, on the contrary, it flows more slowly, and the embracing pressure is thus released from the fluid under which the substances can be dissolved. By repeating the sequence of compression, coagulation and embracing pressure release as described above, the high quality fine particles (such as fine particles having the pure spherical forms) can be obtained.

### EXAMPLES OF TEST CASES

The following describes the results of the testing that was performed under the test conditions listed below, under which soybeans were crushed into fine particles by performing the method of the present invention using the apparatus of the present invention as shown in FIGS. 36(a) and (b).

Objects being tested: soybeans (containing dry fibers 40 to 300 μm long)

Applied pressure: 4.9 MPa (supplied by Pressure Pump)

Machine: apparatus of the invention shown in FIGS. 36(a) and (b) made of SUS316

cylindrical type having the length of 230 mm

external diameter of 140 mm and internal diameter of 70 mm

fluid flow path (a collection of plate members): 110 mm×50 mm made of SUS316

a collection of plate members having the fluid flow path formed on the front and rear sides (FIG. 35(a)): two sets

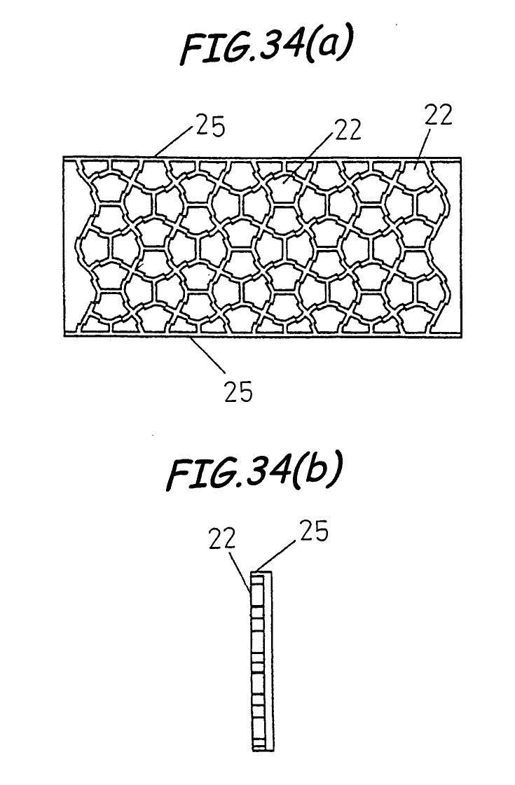

a collection of plate members having the fluid flow path formed on the front side (FIG. 34(a)): two sets

Electron microscope: power of 1000 (one scale division=1.538 micron)

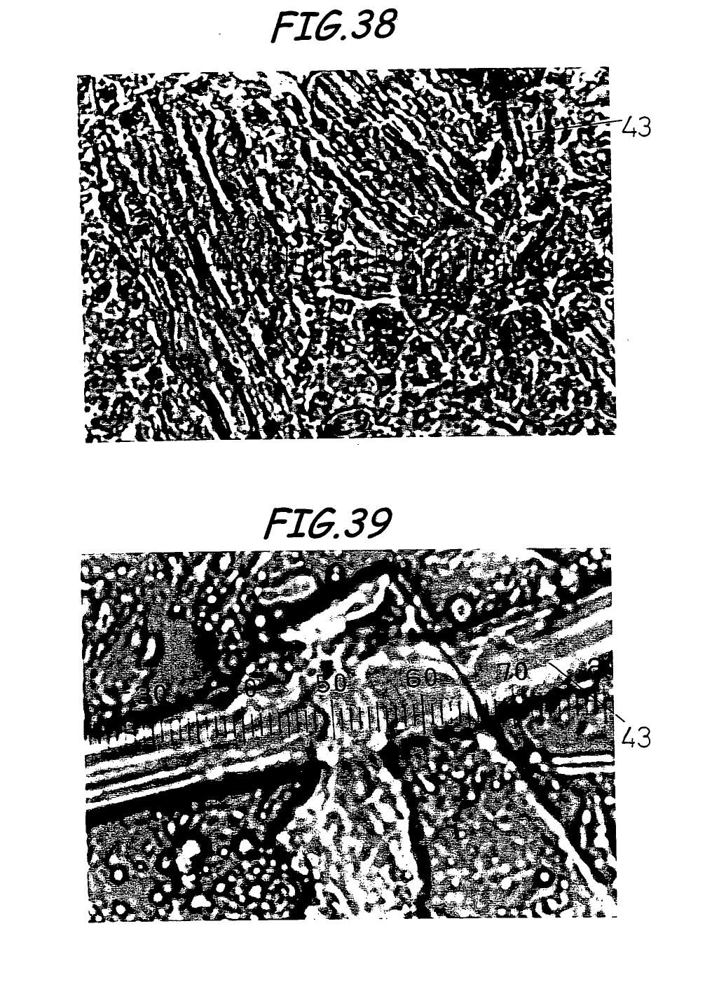

FIGS. 38 through 42 represent the microscopic pictures showing how the soybean fibers, which were obtained at time intervals of one, three and five minutes, can appear through the optical microscope, in which 10 liters of water was added to 2.0 kg of soybeans in their previously crushed forms, and the resulting mixture was fed under the applied pressure into the apparatus by means of the pressure pump, and the testing was made at the above time intervals under the conditions listed above by circulating the soybeans through the apparatus.

In FIG. 38, reference numeral 43 refers to the state of the soybean fibers before being fed under the applied pressure.

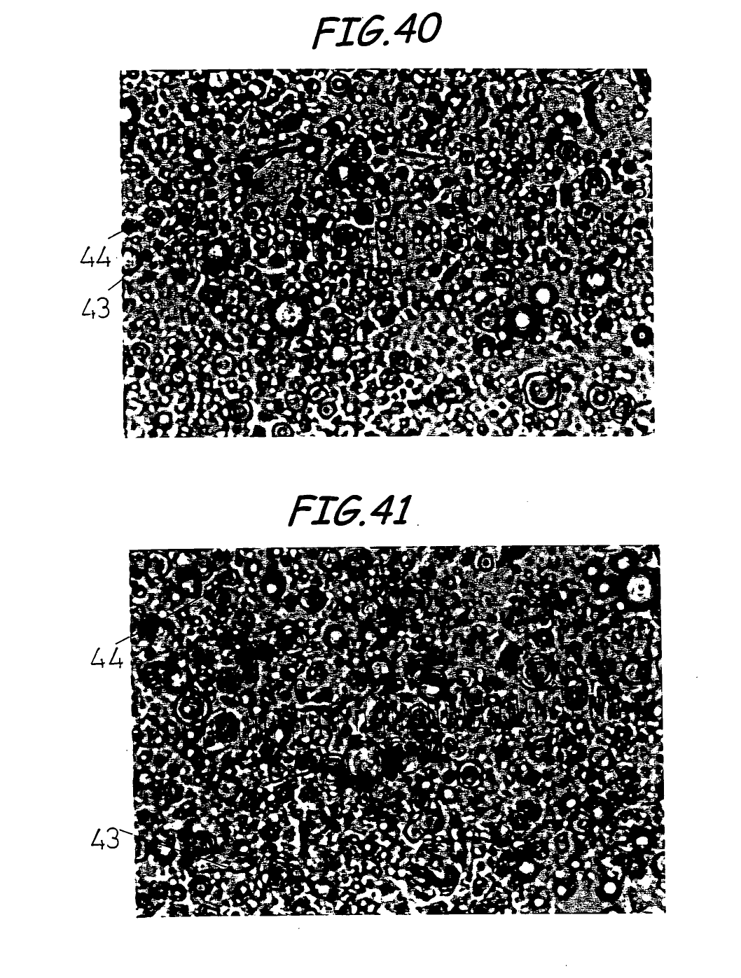

FIG. 39 represents the partially enlarged microscopic picture that shows that the soybeans 43 contain numerous fibers of different sizes.

FIG. 40 represents the microscopic picture showing the state of the soybean fibers at the elapse of one minute after the soybeans were fed under the applied pressure. The picture shows that much of the soybean fibers have been crushed into fine particles, with some soybean fibers still remaining.

FIG. 41 represents the microscopic picture showing the state of the soybean fibers at the elapse of three minutes after the soybeans were fed under the applied pressure. The picture shows that the soybean fibers have mostly been crushed into fine particles, which are in nearly uniform forms.

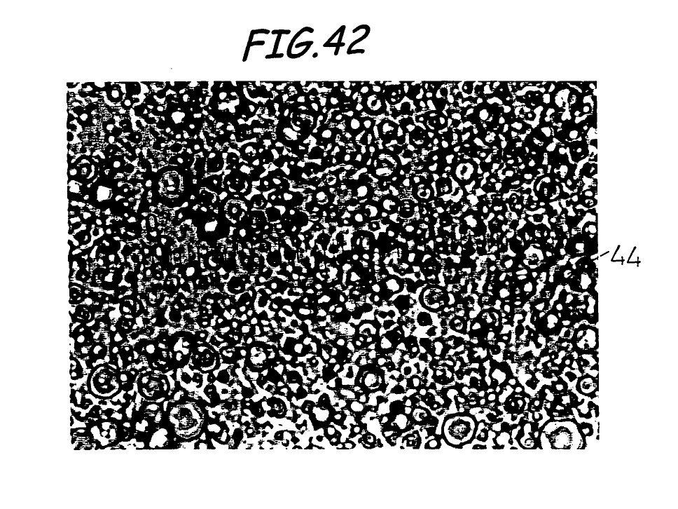

FIG. 42 represents the microscopic picture showing the state of the soybean fibers at the elapse of three minutes after the soybeans were fed under the applied pressure. The picture shows that all of the soybean fibers have been crushed into fine particles, with no soybean fibers remaining and with all fine particles 44 having the spherical forms.

None of the methods and apparatuses that are known in this field permit a fluid of fibrous powders to be crushed into fine particles having the nearly pure spherical forms, even by any of the mixing, stirring, shearing, breaking and like means. By using the methods and apparatuses of the present invention, it is possible to crush various types of fibrous materials into fine particles having nearly pure spherical forms.

### Embodiment 3

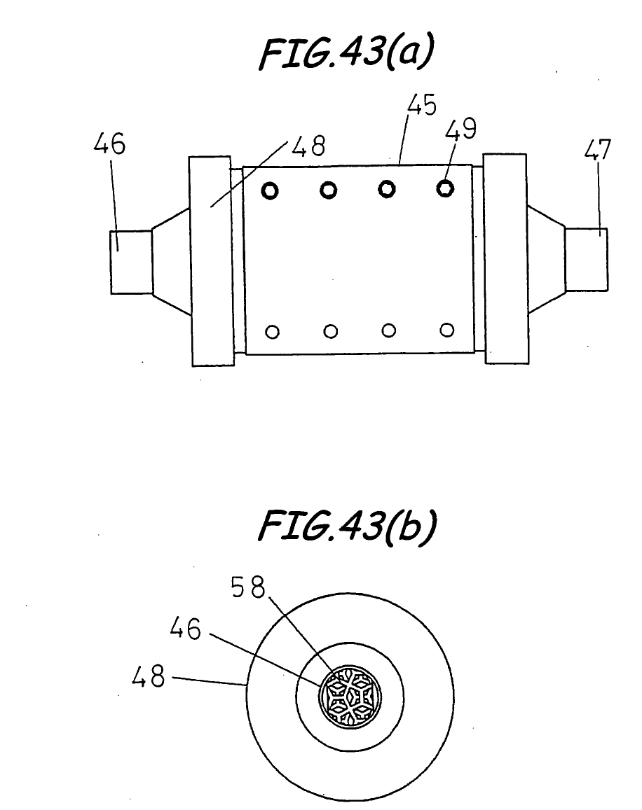

Referring now to FIG. 43, another embodiment of the apparatus for mixing and/or crushing substances into fine particles in accordance with the present invention is described. FIG. 43(a) is a front view of the apparatus including the components constructed in accordance with the present invention, and FIG. 43(b) is a side elevation.

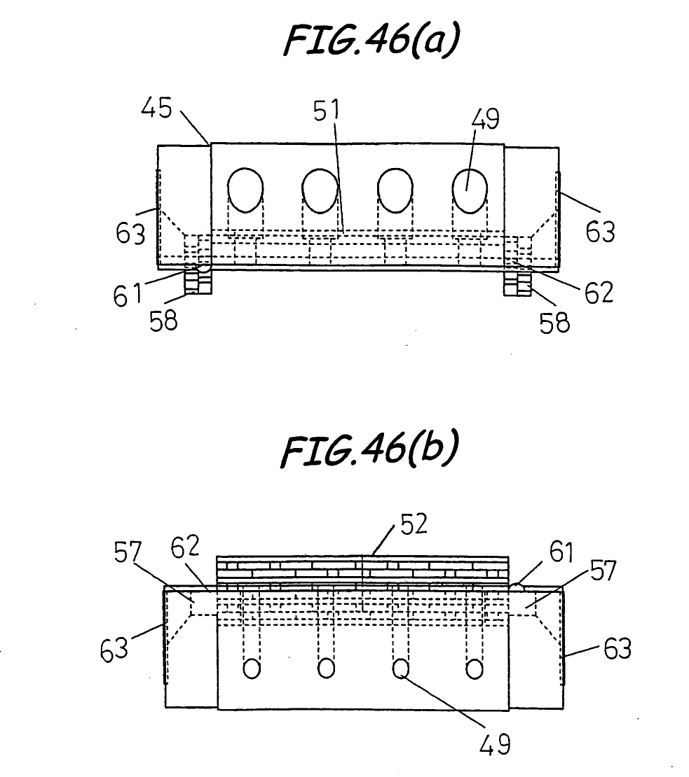

The apparatus comprises a cylindrical casing 45, and a cover 48 having an inlet 46 may be mounted to the inlet side of the casing 45 and a cover 48 having an outlet 47 may be mounted to the outlet side of the casing 45. The cylindrical casing 45 has a plurality of bolt insertion holes 49 provided at regular intervals on the upper and lower portions. The cylindrical casing 45 has the divisible construction that permits the casing 45 to be divided into two parts in the horizontal axial direction as shown in FIGS. 45(a), (b) and FIGS. 46 (a), (b).

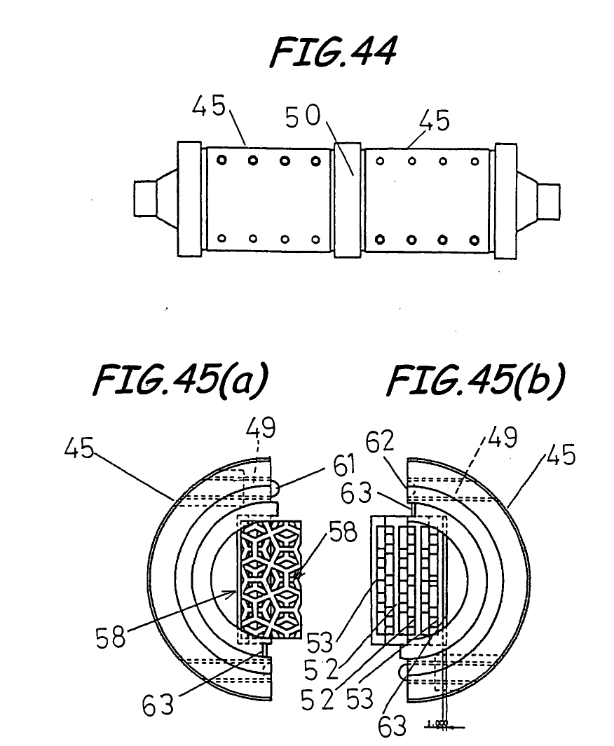

The apparatus may include two parts that can be connected together by connectors 50, as shown in FIG. 44.

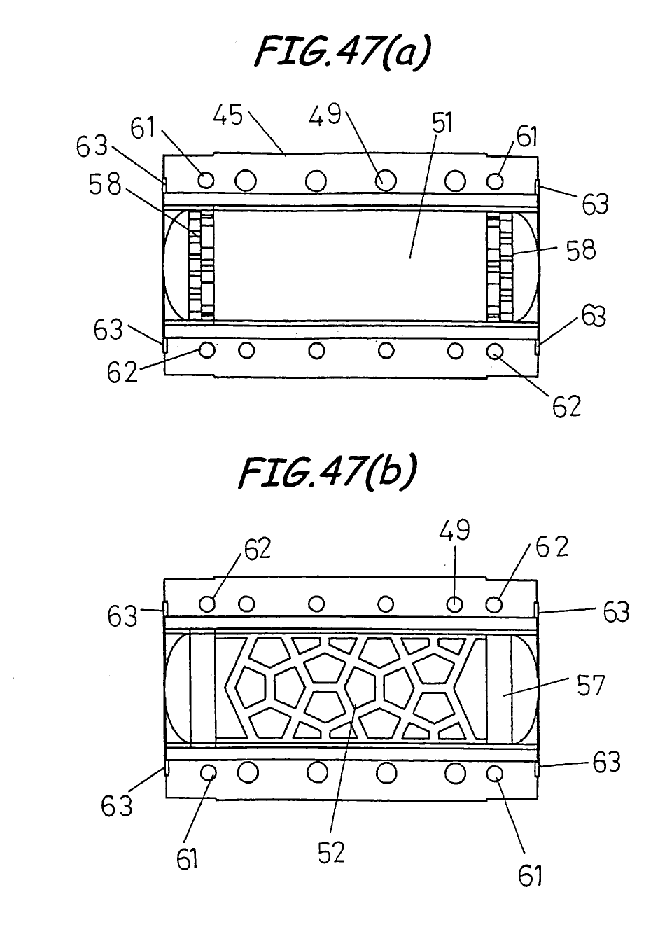

FIGS. 45(a), (b), FIGS. 46(a), (b), and FIGS. 47(a), (b) show how the apparatus shown in FIGS.(a), (b) is divided into two parts in the horizontal axial direction.

FIGS. 45(a), (b) represent a side elevation with some non-critical parts being not shown, in which FIGS. 45(a) represents a side elevation of the inlet side 46 or outlet side 47, and FIG. 45(b) represents a cross section in the central portion of the cylindrical casing 45 as viewed from the lateral side, with some non-critical parts being not shown.

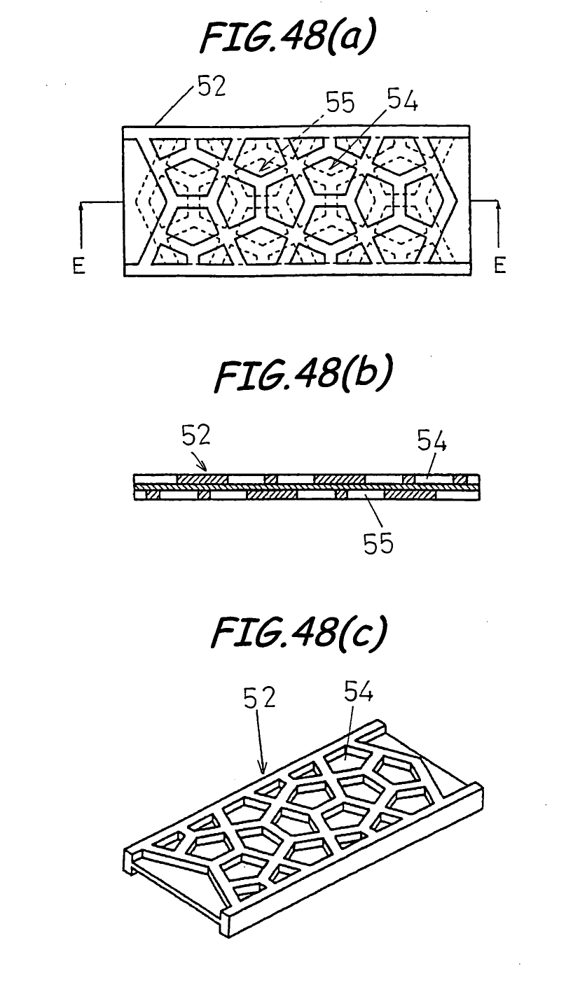

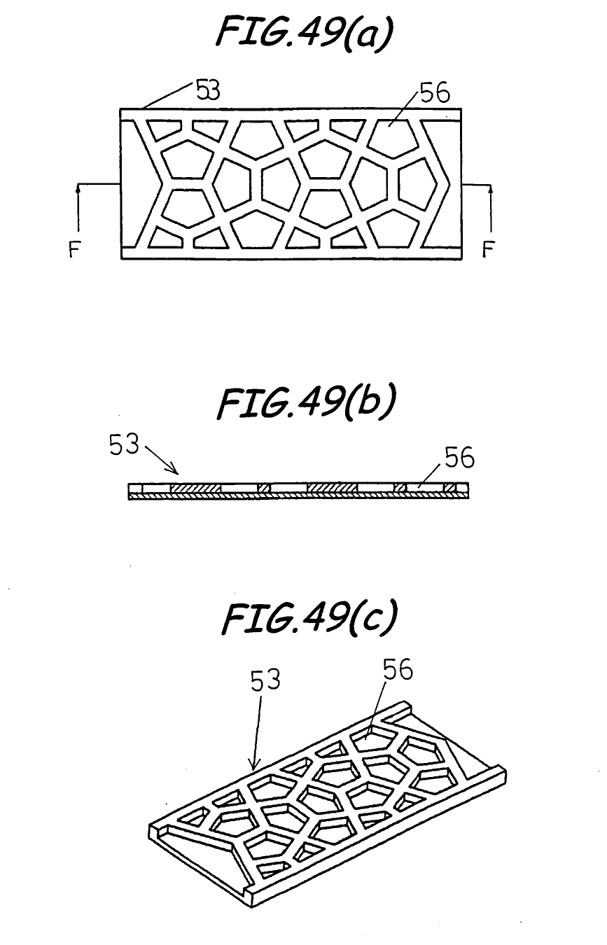

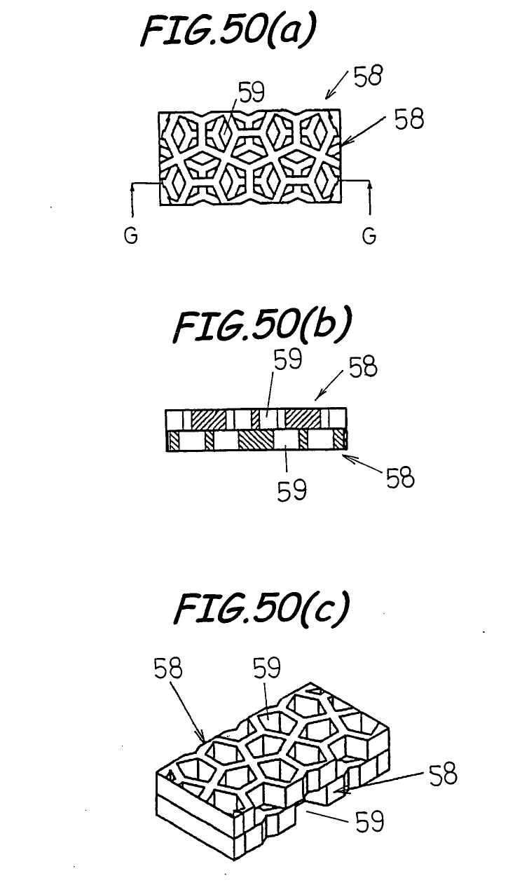

FIGS. 46(a), (b) correspond to the plan view, in which FIG. 46(a) illustrates how the frame member 58 to be described in FIGS. 50(a) through (c) can be mounted and removed, and FIG. 46(b) illustrates the structural member 52 forming the fluid flow path to be described in FIG. 48(a) through FIG. 49(c) can be mounted and removed.

FIGS. 47(a), (b) correspond to the front view, in which FIG. 47(a) illustrates the portion of the fluid flow path that can be formed by the frame member 58 to be described in FIGS. 50(a) through (c), and FIG. 47(b) illustrates the portion of the structural member 52 to be described in FIG. 48 (a) through FIG. 49(c).

The cylindrical casing 45 has a first recess 51 formed on the inner circumferential wall (FIG. 46(a)).

A first plate member 52 shown in FIGS. 48(a) through (c) and a second plate member 53 shown in FIG. 49(a) through (c) may be fitted in the first recess 51 and secured therein.

It may be seen from FIGS. 48(a), (b) that the first plate member 52 has a plurality of pentagonal cells 54, 55, each having its front side open, arranged on each of the opposite sides thereof, respectively. In the embodiment now described, as shown in FIGS. 48(a), (b), the cells 54, each having its front side open, on the upper side of the first plate member 52 and the cells 55, each having its front side open, on the lower side of the first plate member 52 are all similar in the shape, but the cells 54 on the upper side are arranged in alternate positions and in different positions by an angle of 180 degrees, with regard to the cells 55 on the lower side.

The second plate member 53 may have a plurality of pentagonal cells 56, each having its front side open, on at least one side thereof. In this embodiment, the cells 56 are arranged on the upper side of the second plate member 53, as shown in FIGS. 49(a), (b).

It may be seen from FIG. 45(b) that the first plate member 52 and second plate member 53 are arranged adjacently to each other such that the respective cells 54, 55, 56 on the first and second plate members can face opposite each other, with the respective cells 54, 55, 56 being placed in alternate positions with respect with each other and with each of the cells on one plate member communicating with at least one or more of the cells on the other plate member.

As described above for the first plate member 52, the cells 54 on the upper side and the cells 55 on the lower side are placed in alternate positions with regard to each other, with each of the cells 54 being displaced by the angle of 180 degrees with regard to each corresponding one of the cells 55. As shown in FIG. 45(b), a fluid flow path that passes through the cells 54, 55 may thus be formed on the area where the two adjacent first plate members 52, 52 are placed adjacently to each other.

A fluid flow path may also be formed on the area where the first plate member 52 and the second plate member 53 are placed adjacently to each other, as shown in FIG. 45(b). To this end, it is necessary that the pentagonal cells 56, each having its front side open, on the second plate member 53 may be placed in alternate positions, or displaced by a predetermined angle of 45 degrees, for example, with regard to any of the cells 54, 55 on the opposite sides of the first plate member 52 when they are placed to face opposite each other.

It may be appreciated that the embodiment shown in FIG. 45(b) may be varied such that two adjacent second plate members 53, 53 may be placed back-to-back (the lower side in FIG. 49(b)), and a fluid flow path may be formed by placing a first plate members 52 on each of the opposite sides of the combined second plate members 53, 53 such that first plate members 52, 52 can sandwich the second plate members therebetween.

This embodiment may further be varied to meet the particular properties of substances being processed as well as the particular processing requirements such as mixing and/or crushing into flue particles.

The cylindrical casing 45 also has a second recess 57 on the inner circumferential wall thereof (FIG. 46(b)).

Frame members 58, 58 that will be described in FIGS. 50(a) through (c) may be fitted in the second recess 57 and secured therein. The frame members 58 form a plurality of openings 59 arranged like a honeycomb and communicating with each other in the axial direction of the cylindrical casing 45. A plurality of frame members 58 are provided, and any two adjacent frame members 58 may be arranged one over the other in the direction perpendicular to the axial direction of the cylindrical casing 45, such that the openings 59 on one frame member can be placed in alternate positions with regard to the corresponding openings 59 on the other frame member. In any of the embodiment shown in FIG. 45(a) and FIGS. 50(a) through (c), the openings 59 have the pentagonal shape.

FIG. 48 represents the first plate member 52, in which FIG. 48(a) corresponds to the front view, FIG. 48(b) corresponds to the section along the line E-E, and FIG. 48(c) corresponds to the perspective view. The first plate member 52 has a plurality of pentagonal cells 54, 55, each having its front side open, on the opposite sides thereof As described, the cells 54 on the upper side are placed in alternate positions and are displaced by an angle of 180 degrees, with regard to the cells 55 on the lower side.

It should be understood that the present invention is not limited to the first plate member 52 described in this embodiment, but the sizes of the cells 54, 55, each having its front side open, as described and shown, may be changed, and the number of cells 54, 55 may be increased or decreased, depending upon the particular properties or mixture ratio of the substances being processed, such as mixing and/or crushing.

FIG. 49 represents the second plate member 53, in which FIG. 49(a) illustrates the front view, FIG. 49(b) illustrates the sectional view along the line F-F, and FIG. 49(c) illustrates the perspective view.

The shape of the cells 56, each having its front side open, on one side of the second plate member 53 is similar to the shape of the cells 54, 55 on the first plate member 52, but as described earlier, the positions of the cells 56, each having its front side open, on the second plate member 53 are displaced by an predetermined angle, such as 45 degrees, with regard to the positions of the corresponding cells 54, 55 on the first plate member 52, and the cells 56 are placed in alternate positions with regard to the corresponding cells 54, 55 when the former 56 is placed to face opposite the latter 54, 55.

Similarly, it should be understood that the present invention is not limited to the second plate member 53 described in this embodiment, but the sizes of the cells 56, each having its front side open, as described and shown, may be changed, and the number of cells 56 may be increased or decreased, both depending upon the particular properties or mixture ratio of the substances being processed, such as being mixed and/or crushed.

FIG. 50 represents how a plurality of frame members 58 that are placed one over another can be fitted easily in the second recess 57 and secured therein, in which FIG. 50(a) illustrates the front view, FIG. 50(b) illustrates the sectional view along the line G-G, and FIG. 50(c) illustrates the perspective view.

The frame member 58 has a plurality of pentagonal openings 59, 59 extending through the frame member 58. Any two adjacent frame members 58, 58 are placed one over the other such that the openings 59 on one of the frame members facing opposite each other can be placed in alternate positions with regard to the corresponding openings 59 on the other frame member.

The frame member 58 has the external shape that is similar to the shape of the second recess 57 on the inner circumferential wall of the cylindrical casing 45. This makes it easy to fit the frame member 58 in the recess 57 or to be removed from the recess 57.

It should be understood that the sizes of the openings 59 that can be provided on the frame member may be changed, and the number of the openings 59 may be increased or decreased, both depending on the particular properties and mixture ratios of the substances being processed, such as mixed and/or crushed.

In FIGS. 45(a) through 47(b), the part denoted by 61 refers to a positioning projection, and the part denoted by 62 refers to a positioning recess. As described, the cylindrical casing 45 has the divisible construction that permits the cylindrical casing 45 to be divided into two parts in the axial direction thereof, Those two parts may be assembled by engaging the first plate member 52 and second plate member 53, and engaging the frame members 58, 58 with the second recess 51, followed by engaging the positioning projection 61 with the positioning recess 62 and then inserting bolts into the bolt insertion holes 49. Thus, this facilitates the disassembling and reassembling of the cylindrical casing 45 for the maintenance services.

In FIG. 45(a) through FIG. 47(b), the part denoted by 63 refers to a packing that provides the sealing function.

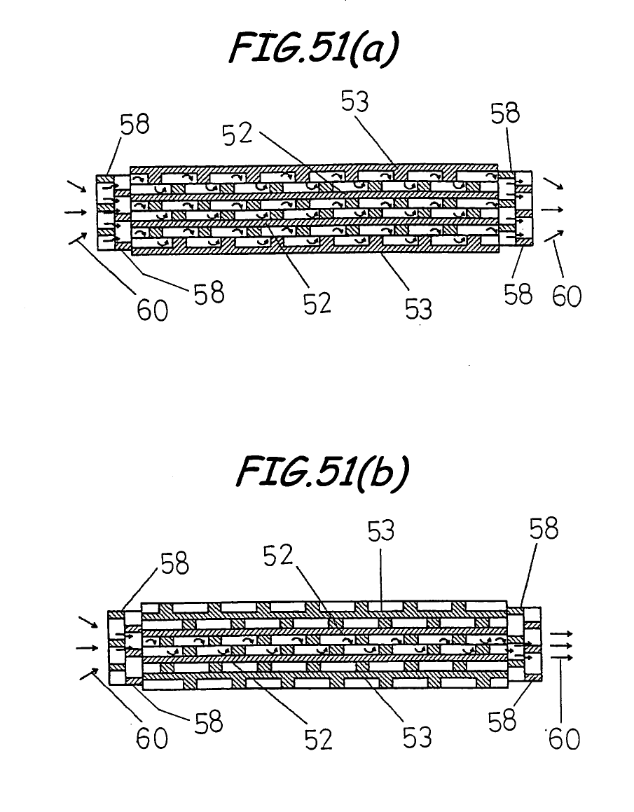

FIG. 51(a) is a schematic cross sectional diagram that illustrates how a fluid 60 of substances being processed, such as being mixed and/or crushed into fine particles, can flow through the fluid flow path formed by the frame members 58, 58 mounted inside the cylindrical casing 45 and through the fluid flow path formed by the first plate member 52 and second plate member 53, circulating through those fluid flow paths.

The fluid 60 may be delivered into the cylindrical casing 45 through the inlet 46 of the cover 48 by using any appropriate delivery means. Then, the fluid 60 may flow through the fluid flow path formed by the openings 59, 59 on the frame members 58, 58, producing some dispersing action.

Then, the fluid 60 flows through the fluid flow path formed by the first plate member 52 and second plate member 53, hitting against the walls forming the cells 54, 55, 56 and repeating the dispersing, swirling and reversing actions. During those repeated actions, the fluid may have the process of mixing and/or crushing the substances in the fluid into fine particles, flowing toward the outlet 47.

On the side of the outlet 47, the fluid 60 may flow through the fluid flow path formed by the openings 59, 59 on the frame members 58, 58 until the fluid reaches the outlet 47 on the cover 48 through which it goes out.

FIG. 51(b) represents the schematic sectional diagram showing that the placement of the first plate member 52 and second plate member 53 one over the other is reversed from the placement shown in FIG. 51(a) and how the fluid 60 can flow the fluid flow path in this case.

### Embodiment 4

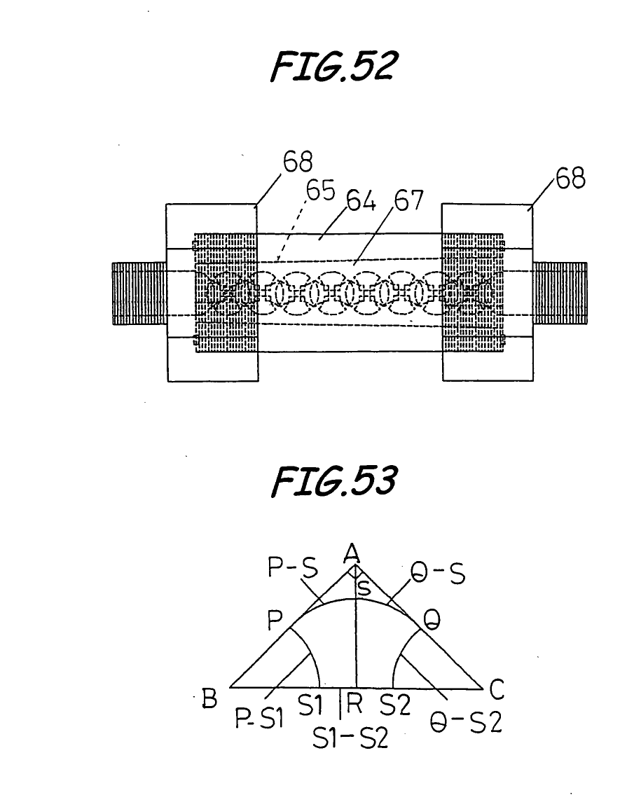

FIGS. 52 through 55 illustrates another embodiment of the apparatus according to the present invention, wherein it includes the cells that are formed so that they can form the fluid flow paths in the manner described in FIGS. 30 through 33(b), and includes the plate members having such cells, each having its front side open.

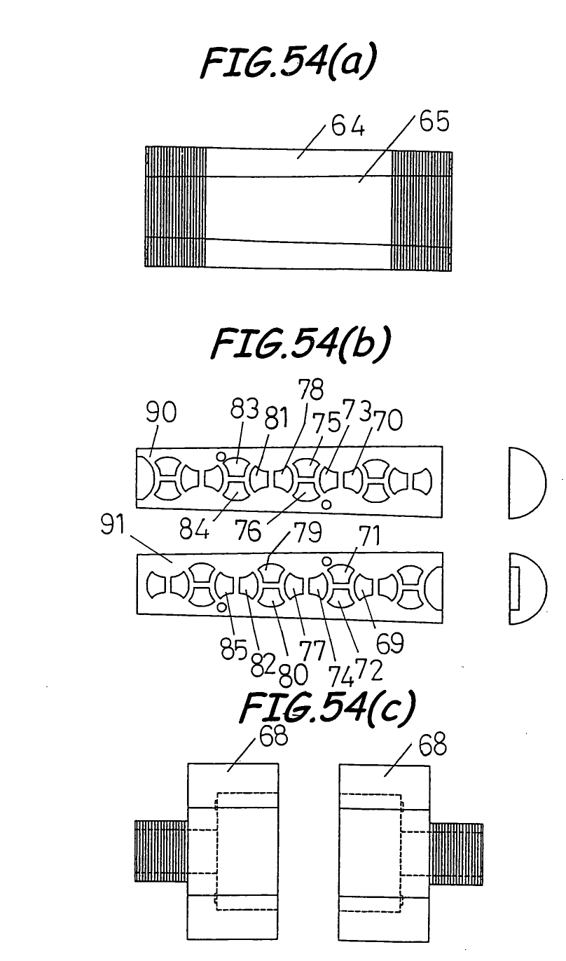

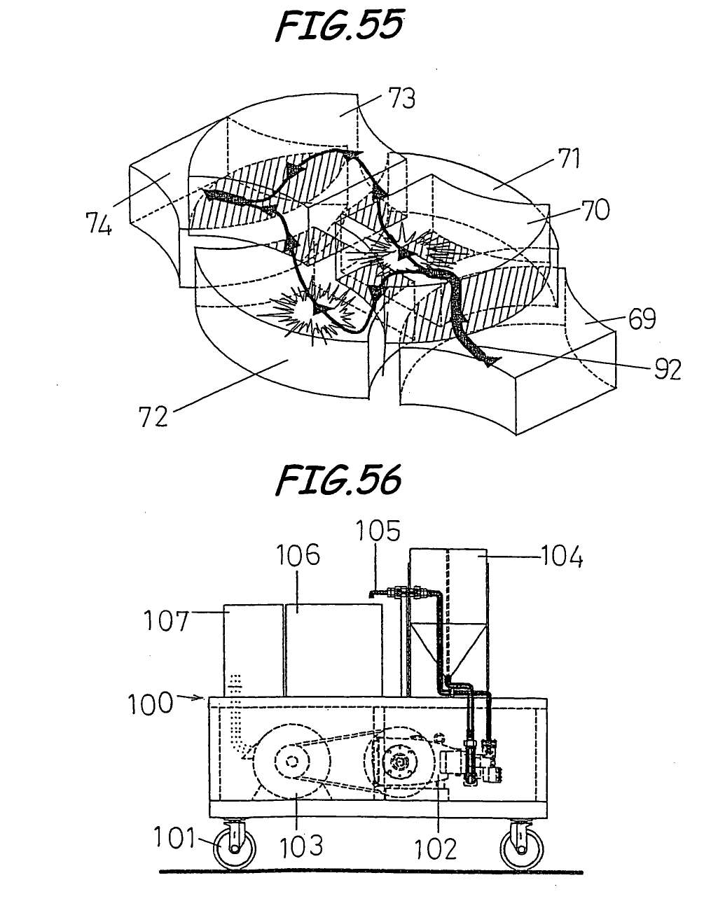

FIG. 52 represents the front perspective view with some non-critical parts being not shown, and FIGS. 54(a) through (c) represent the exploded view of FIG. 52.

The apparatus shown in FIG. 52 includes a cylindrical casing 64 having the external cylindrical shape and having a hollow portion 65 therein. The hollow portion 65 may accept a fluid flow unit 67, which is so completely fitted inside the hollow portion 65 that it can prevent the short path from occurring.

A connecting screw 68, 68 may be removably mounted on each of the opposite ends of the cylindrical casing 64 that may be used to connect the apparatus and any other external devices to the cylindrical casing 64. Those connecting screws 68 may also serve to prevent the fluid flow unit 67 inside the hollow portion 65 from projecting out of the cylindrical casing 64.

The fluid flow unit 67 includes a plate member 90 having a plurality of cells 70, 72, 75, 76, 78, 81, 83, 84, each having its front side open, arranged on one side thereof, and a plate member 91 having a plurality of cells 69, 71, 72, 74, 77, 79, 80, 82, 85, each having its front side open, arranged on one side thereof, wherein the plate member 90 and plate member 91 are placed adjacently to each other such that the cells on one plate member can face opposite the corresponding cells on the other plate member, with the cells facing opposite each other being placed in alternate positions and with each of the cells on one plate member communicating with at least one or more of the cells on the other plate member. For example, the fluid flow unit 67 may be formed by placing the plate members 90 and 91 adjacently to each other such that a cell 69 having its front side open on the plate member 91 can communicate with a cell 70 having its front side open on the plate member 90, the cell 70 having its front side open on the plate member 90 can communicate with cells 71, 72 having its front side open on the plate member 91, and the cells 71, 72 having its front side open on the plate member 91 can communicate with a cell 73 having its front side open on the plate member 90.

Thus, the fluid flow unit 67 may be formed by the sequence of the plurality of cells 69 through 85 on one plate member, each of which communicates with at least one or more of the cells, each having its front side open, on the opposite plate member.

FIG. 53 illustrates a particular geometric shape that may be used as the basis for defining the shape of each of the cells 69 through 85, etc. on the plate members 90, 91 forming the fluid flow unit 67. Each of the cells 69, etc. has the shape including a base defined as a straight line and other sides defined as arc curves.

A right-angled isosceles triangle ΔABC is given, where the triangle has a vortex A, a base B-C, and hypotenuses A-B and A-C. Then, arbitrary points P, Q may be set anywhere along the hypotenuses A-B and A-C, and an arbitrary point S may be set anywhere along the perpendicular between the vortex A and base B-C other than points A, R. Then, arc curves P-S and Q-S connecting the arbitrary points P, Q on the hypotenuses and the arbitrary point S on the perpendicular are turned about P, Q, respectively, such that P-S may be turned clockwise through an angle of 90 degrees, and Q-S may be turned anticlockwise through an angle of 90 degrees. Then, points that abut on the base B-C may be designated S1, S2. The shape that results is surrounded by arc curves P-S, Q-S, arc curves P-S1, Q-S2, and line S1-S2 connecting points S1, S2.

The line P-S and Q-S may have different types of lines, such as straight line, curve, sine curve arc line, broken line and the like.

It is therefore important that the area of the shape surrounded by the line segments P-S-Q-S2-S1-P formed as described above should be equal to one half the area of the right-angled isosceles triangle ABC. In this way, the cells can be arranged on the plate members 90, 91 without any gaps being produced between any adjacent cells. As long as this condition is met, the arbitrary points P, Q, S may be placed on any locations on the sides A-B, A-C and on the perpendicular A-R.

It should be noted that the line connecting between point S1 and point R on the base may be a straight line, and the line connecting between point S2 and point R on the base may be a curved line.

It should also be noted that the fluid flow unit 67 may have the column shape that includes conical portions on the opposite ends thereof that may be separated from each other, and may be recombined into one unit by means of guide pins provided on appropriate locations

In addition, it is desirable that the cells 69 through 85 etc. on the plate members 90, 91 forming the fluid flow unit 67 should be placed in the respective positions that are displaced relative to each other by rotating one plate member relative to the other plate member through an angle of 180 degrees, as shown in the upper and lower portions of FIG. 54(b), when the plate members 90, 91 are placed adjacently to each other such that the open front sides of the cells on one plate member can face opposite those of the cells on the other plate member. In this way, the cells 70, 73, 75, 76, 78, 81, 83, 84 on one plate member 90, and the cells 69, 71, 72, 77, 79, 80, 82, 85 on the other plate member 91 that face opposite the cells on the one plate member 90 can divide the respective front space into a number of space portions, and each of the cells on each of the plate members can communicate with at least one or more of the cells on the other plate member.

FIG. 55 is a schematic diagram illustrating how the fluid 92 of substances being processed, such as being mixed and/or crushed into fine particles, can flow through the fluid flow unit 67. By referring to FIG. 55, the flow of the fluid is explained below.

The fluid 92 first enter a cell 69 on the lower plate member 91, and then enters a cell 70 on the opposite plate member 90. Then, the flow 92 is divided into two flows that flow into cells 71, 72 on the opposite plate member 91. The two flows rejoin each other into a single flow that enters a cell 73 on the opposite plate member 90. The fluid that exits the cell 73 then enters a cell 74 on the opposite plate member 91, where the flow is divided into two flows that enter cells 75, 76 on the opposite plate member 90. Then, the two flows rejoin each other into a single flow that enters a cell 77 on the opposite plate member 91.

While the fluid 92 is flowing through the cells as described above, the fluid 92 goes through the continuous process of dispersing, concentrating, rejoining, compressing by pressure, and releasing from pressure, and this process is repeated until the substances can finally be mixed and/or crushed into ultrafine particles or molecules.

### Embodiment 5

FIG. 56 is a general arrangement diagram illustrating one example of the apparatus described above in accordance with the embodiments 1 through 4, in which the apparatus may be used to crush soybeans into ultrafine particles. The apparatus generally shown by 100 is equipped with casters 101 that permit the apparatus to be traveling. The apparatus 100 contains a drive motor 103 on the bottom for driving a pressure pump 102, and includes an inverter 107. In addition, a hopper 104 is mounted on the top of the apparatus, through which soybeans may be delivered into the apparatus, and a return vessel 106 is provided near the outlet 105 of the apparatus 100, into which the soybeans just processed, such as crushed into ultrafine particles, may be collected.

The operation is now described. When a fluid of soybeans is placed into the hopper 104, the fluid goes through the delivery piping where the fluid is placed under the appropriate pressure supplied by the pressure pump 102, going into the apparatus 100 through an inlet (not shown). Then, the fluid flows through the fluid flow paths in the fluid flow unit 57 described in FIG. 52, and FIGS. 54(a), (b) and in the manner described in FIG. 55. When entering the cells 69 through 85 under the applied pressure, the soybeans may be compressed strongly by the strong pressure, followed by being released immediately from the compression. Thus, the soybeans may continue to be broken into ultrafine particles by the internal and external pressures released by exploding themselves, and the ultrafine particles may be collected into the return vessel 106 through the outlet 105. The process of the soybeans continuing to be broken into ultra-fine particles by the internal and external pressures released by exploding themselves when the soybeans are flowing through the fluid flow paths as described in FIG. 55 may be explained by the so-called “dissipation theory”.

### Embodiment 6

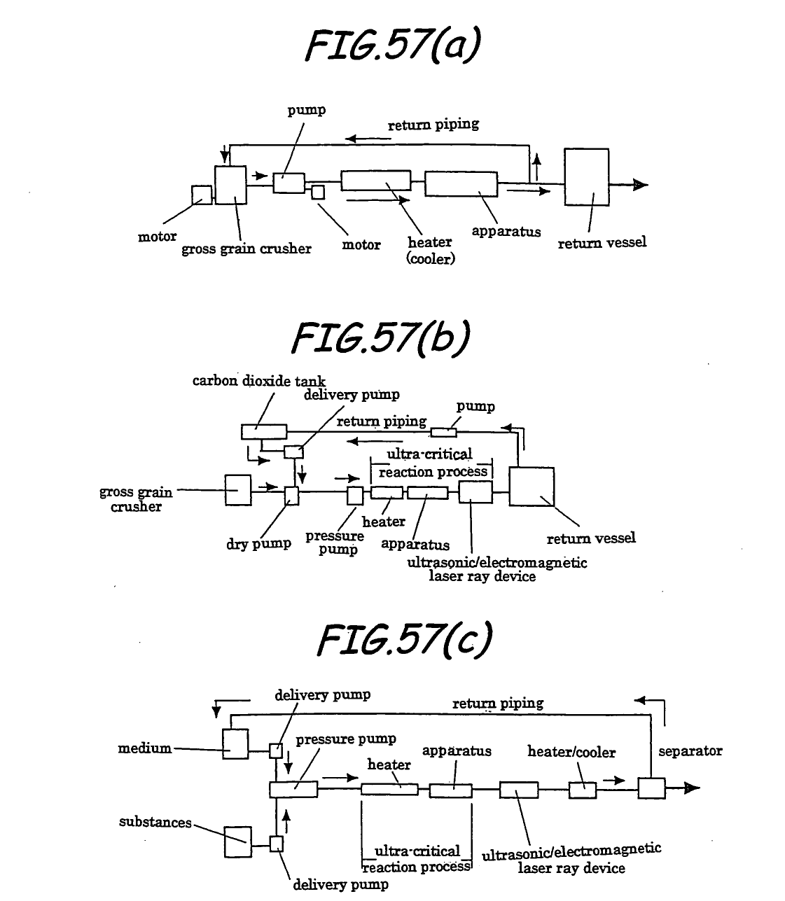

FIGS. 57(a) through (c) are block diagrams illustrating the process of the method according to several embodiments of the present invention that may be implemented on the apparatus described above in the embodiments 1 through 4.

FIG. 57(a) is a schematic block diagram illustrating one example of the method that includes the steps of crushing wet-type substances into fine particles that may be implemented on the apparatus of the invention. The process is now described below by referring to FIG. 57(a).

Raw substances being processed, such as being mixed and/or crushed into fine particles, may first be supplied into a gross particle crusher where the raw substance are crushed into grossly crushed particles that may be delivered into a heater by a delivery pump, from which the grossly crushed particles may be delivered into the fluid flow paths in the apparatus of the present invention. Passing through the fluid flow paths, the particles may be crushed into finer particles having the desired particle sizes and then be collected in the storage vessel. A filter that is disposed between the apparatus and the storage vessel may filter the particles, and those particles that have not passed through the filter may be returned to the gross particle crusher where they may be crushed into finer particles again as described above and then collected in the storage vessel. The particles in the storage vessel may be delivered to any following step (finishing line).

FIG. 57(b) is a schematic block diagram illustrating one example of the method that includes the steps of crushing substances into fine particles by causing the substances to react under the continuous ultracritical processing conditions using the carbon dioxide that may be implemented by the apparatus of the present invention in combination with the ultrasonic waves/electromagnetic wave/laser ray illuminating devices. The process is now described below by referring to FIG. 57(b).

Raw substances that have previously been crushed into gross particles may be mixed with any extracted solvent such as carbon dioxide through the delivery pump and dry-type pump, and the resulting mixture may be brought to the appropriate pressure and temperature levels by means of the pressure pump and heater that would cause the mixture to be placed under the continuous ultracritical conditions. Then, the mixture may be delivered under the applied pressure into the cylindrical casing in the apparatus. While flowing through the fluid flow paths, the mixture may be placed under the continuous ultracritical conditions, under which the mixture may be crushed into ultrafine particles. The substances thus crushed into the ultrafine particles may then be exposed to the ultrasonic waves, electromagnetic waves, laser rays, etc. so that they can react chemically or can be dissolved.

The final product thus obtained may be collected into the return vessel, and the extracted solvent in its liquefied state may be delivered to the pressure control valve (not shown), through which the solvent may be gasified for recycling.

FIG. 57(c) is a schematic block diagram illustrating one example of the method that includes the steps of crushing substances into fine particles by causing the substances to react under the continuous ultracritical processing conditions using some types of solvents that may be implemented by the apparatus of the present invention in combination with the ultrasonic waves/electromagnetic wave/laser ray illuminating devices. The process is now described below by referring to FIG. 57(c).

Any extracted solvent in its liquid state and the substances being processed, such as being dissolved, may be mixed together through the delivery pump, and the resulting mixture may be brought to the appropriate pressure and temperature through the heater pump that would cause the mixture to be placed under the ultracritical conditions and may then be delivered under the applied pressure into the cylindrical casing in the apparatus. While flowing through the fluid flow paths, the mixture may be placed under the continuous ultracritical conditions, under which the mixture may be crushed into ultrafine particles. The substances thus crushed into the ultrafine particles may then be exposed to the ultrasonic waves, electromagnetic waves, laser rays, etc. so that they can react chemically or can be dissolved. Through the sequence of the steps described above, the inter-molecule collision and dissolving may occur continuously, which may promote the chemical reaction. The substances that have thus been dissolved may be delivered to the cooler, and is then delivered to the gas-liquid separator where the gas and liquid are separated, with the gas being processed to become harmless and the extracted solvent in its liquid state being returned through the return piping to the solvent vessel for recycling.

### Embodiment 7

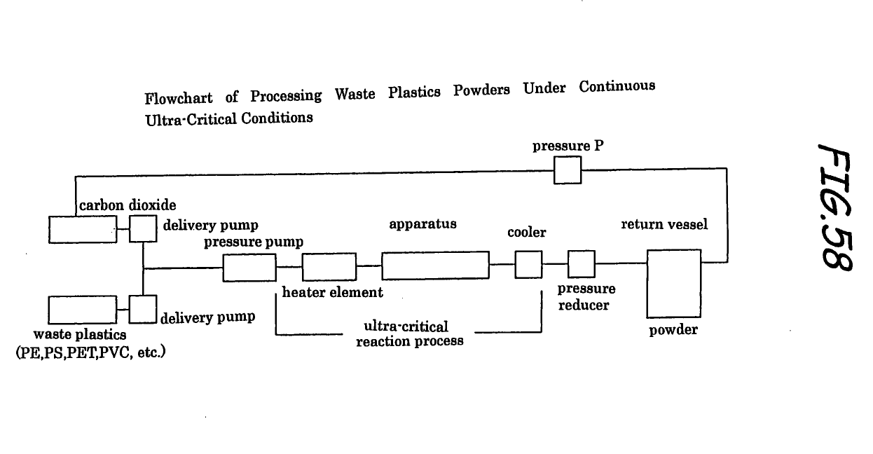

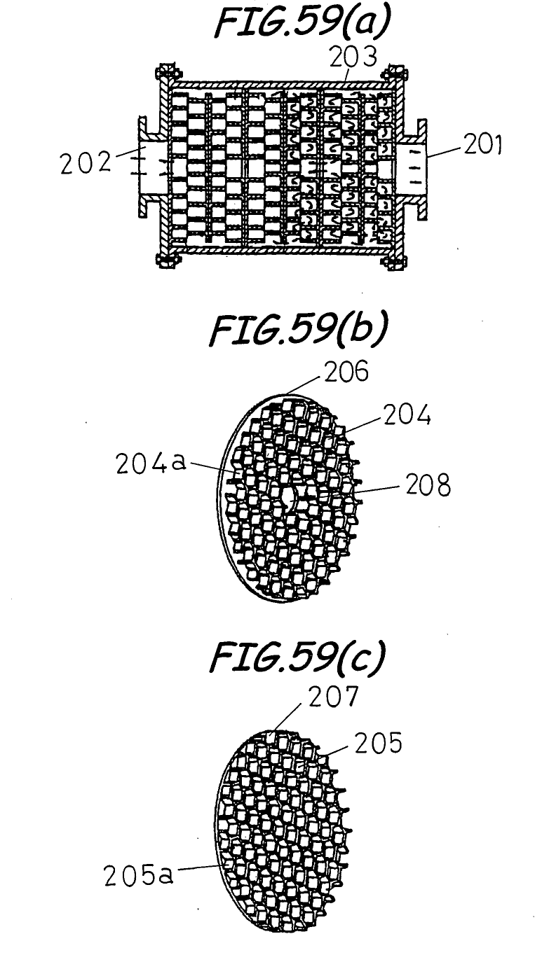

FIG. 58 is a block diagram illustrating the process of the method of the present invention that may be implemented on the apparatus described above in the embodiments 1 through 4.

The waste plastics that have previously been crushed into gross particles and the carbon dioxide that is used as an extracted solvent and/or for oxidization reaction and hydrolysis may be delivered together under the applied pressure toward the inlet of the cylindrical casing through the delivery pump and pressure pump, and may then be heated by any heating elements such as a heater. At this moment, the substances should be placed under the ultracritical conditions for the carbon dioxide under which they should be maintained at the pressure of 7.38 MPa and the temperature of

A fluid of the grossly crushed waste plastics that is placed under the ultracritical conditions may then be delivered under the applied pressure into the fluid flow paths from the inlet of the cylindrical casing, through the fluid may flow under the applied pressure toward the outlet.

While flowing through the fluid flow paths within the cylindrical casing, the grossly crushed waste plastics may be placed under the continuous ultracritical conditions under which they may be crushed into finer particles.

Those substances that have thus been processed and gone out of the outlet may be passed to the cooler and pressure reducer where the substances may be separated into plastics and powders. The powders may be collected into the return vessel, with the gas being returned for recycling.

In this embodiment, the carbon dioxide is used as the extracted solvent and/or for the oxidization reaction and hydrolysis, but it should be understood that any other type of extracted solvent that the carbon dioxide may also be used as long as such extracted solvent can be used when the substances are crushed into fine particles while they are placed under the critical and ultracritical processing conditions.

The waste plastics that may be crushed into fine particles in this embodiment include polyethylene, polystyrene, polyethylene terephthalate and the like. It should be appreciated, however, that the present invention is not limited to such waste plastics, but other substances such as virgin materials, synthetic resin materials and the like may also be processed in the same manner as described above, specifically, may be crushed into fine particles while they are placed under the continuous critical or ultracritical processing conditions.

For those past years, the waste plastics, virgin materials and synthetic resin materials in their pellet forms were frozen, and then were crushed into powders. This is because there was no technology that permits such pellets to be crushed into powders at the normal temperature. But the freezing process was very costly.

When the apparatus of the present invention is used, however, the waste plastics, etc. can be crushed into fine powders at lower costs because it eliminates the need for the freezing process.

When the conventional wet-type crushing machine is used to crush the waste plastics, etc. into fine powders, it is very difficult to separate the product into the powders and gas.

When the method steps shown in FIG. 58 and FIGS. 57(b), (c) are performed by the apparatus of the present invention, the substances can be crushed into fine particles while they are placed under the continuous critical and ultracritical conditions, and the resulting substances can be separated into the powders and the gas continuously and easily.

It may be appreciated from the foregoing description that the apparatus according to any of the embodiments of the present invention provides both the dry-type crushing functions and wet-type crushing functions.

Possible Industrial Uses of the Invention

It may be appreciated from the foregoing description that the apparatus and method according to any of the embodiments of the present invention provides the following advantages that will be described below.

One advantage of the present invention is in that the apparatus includes the fluid flow path that permits a fluid of substances being processed, such as being mixed and/or crushed into fine particles, to have the various actions and effects while the fluid flows through the fluid flow path, including the compression by the applied pressure followed by the instantly explosive release from the compression, compression and dispersion followed by the release from the compression and dispersion, production of turbulent flows within the fluid flow path, embracing pressure followed by release pressure, and the like. Those actions and effects may occur continuously so that the substances can be dissolved into fine particles, as explained by the dissipation theory.

This advantage has the accompanying effect that permits the even fibrous substances to be crushed into fine particles having the pure spherical forms.

Another advantage of the present invention is in that the apparatus includes the cylindrical casing, the first and second structural members and/or frame members each having a plurality of cells thereon, each having its front side open, which may be made of composite metal materials such as combinations of carbon and copper, carbon and aluminum, carbon and magnesium, carbon and tungsten, and carbon and titanium oxide, and minerals such as ceramics and tourmaline that enable those mechanical members to provide the catalyst actions.

This advantage has the accompanying effect that permits the first and second structural members to be made of natural or synthetic resins, on which the cells, each having its front side open and having the particular shape derived from the right-angled isosceles triangle, can be formed with the higher precision.

A further advantage of the present invention is in that a plurality of magnets may be arranged on the outer circumference of the cylindrical casing such that they face opposite each other for providing N polarity and S polarity, wherein the fluid of substances being processed, such as being mixed and/or crushed, can be subdivided into finer particles under the magnetic action, which can be mixed more uniformly.

Still another advantage of the present invention is in that the cylindrical casing has the divisible construction that permits the structural members and/or structural units to be mounted to or removed from the cylindrical casing for forming the fluid flow paths within the cylindrical casing, by separating the cylindrical casing into two parts, and fitting the structural members or structural units in the recesses provided on the inner circumferential wall of the cylindrical casing or removing them from the recesses. This permits the easy assembling and reassembling, and disassembling for the maintenance.

As the cylindrical casing has the construction that allows for the easy mounting and dismounting of the structural members and/or structural units to and from the cylindrical casing for forming the fluid flow paths therein as described above, the structural members and/or structural units may be made of different types of materials so that they can form the fluid flow paths. Thus, the substances can be mixed and/or crushed into fine particles under the optimum processing conditions.

For example, the size, number, and shape of the cells, each having its front side open, which are provided on the structural members for forming the fluid flow paths, as well as the type of materials for the structural members, may be changed, depending on the particular properties and mixture ratio of the substances being processed. Thus, the substances may be mixed and/or crushed into fine particles under the optimum conditions.

For the industrial wastes, for example, they may previously be crushed into gross particles in the form of a fluid, which may then be delivered under the applied pressure, together with a particular gas, such as pure oxygen, into the fluid flow path within the cylindrical casing of the apparatus. Then, the fluid can have the dispersing, colliding and swirling actions repeatedly, while flowing through the fluid flow paths formed by the cells. Those actions can dissolve the molecules bonded in the substances, rendering the substances harmless.

In the apparatus according to any of the embodiments described above, the cylindrical casing, as well as the structural members forming the fluid flow paths, may be made of thermally conducting materials, such as copper, aluminum, carbon and other similar materials. In this case, the apparatus may be used as the heat exchanger, in which the substances may be mixed and/or crushed into fine particles, while the heat produced may be exchanged.

When a fluid of substances being processed, such as being mixed and/or crushed into fine particles, is delivered under the applied pressure into the cylindrical casing, the fluid may flow through the fluid flow paths formed by the cells, each having its front side open, which are arranged to face opposite each other. As the fluid is flowing through the fluid flow path, the fluid may first flow into one cell, from which the fluid may then flow out to two cells, from which the fluid may then flow out to one cell, and so on. This is repeated, in which each time the fluid flows in and out, the fluid may be released instantly from the pressure, causing the fluid to explode outwardly, and may have the strong compression. Thus, the substances may be crushed into ultrafine particles or molecules.

It may be understood from the foregoing description that the apparatus can be operated under the critical or ultracritical conditions for a particular type of substances being processed, such as substances that are hard to be dissolved, more specifically, the industrial wastes, environment polluting substances and the like that might contain dioxins, under which such substances can be dissolved and rendered harmless. More specifically, by using the apparatus under the above conditions, the process of mixing the substances with a particular solvent, crushing the substances into ultrafine particles or molecules, promoting the reaction dissolving and dissolving can be enhanced. Thus, the substance dissolving process can be improved. Furthermore, by using the apparatus in conjunction with the ultrasonic wave illuminating means, electromagnetic wave illuminating means, infrared ray illuminating means and/or far infrared ray illuminating means during the above sequence, the process of crushing the substances into ultrafine particles or molecules and promoting the reaction dissolving can be enhanced further. Thus, the substance dissolving process can be improved much further.

Finally, by using the apparatus under the critical and ultracritical conditions, any foodstuffs and raw medicinal substances can be processed continuously, and the process of deactivating, sterilizing, deodorizing any enzymes or spores contained in those materials can occur efficiently, safely and continuously. This includes the process of controlling the chemical reaction for chemical substances, generating chemical substances, dissolving chemical substances and the like.

What is claimed is:

1. An apparatus for mixing and/or crushing substances into fine particles, which comprises a cylindrical casing having a hollow portion inside it and having an inlet on one end and an outlet on the opposite end, wherein the cylindrical casing includes a fluid flow path inside it that runs across the cylindrical casing for allowing the fluid to flow from the inlet end toward the outlet end and wherein the fluid flow path includes a first structural member having a plurality of first cells thereon, each having its front end open, and a second structural member having a plurality of second cells thereon, each having its front side open, the first structural member and the second structural member being placed adjacently to each other such that the open front sides of the first cells on the first structural member are located to face opposite the open front sides of the second cells on the second structural member, with the first cells on the first structural member being placed in alternate positions with regard to the second cells on the second structural member, and with each of the cells on one structural member communicating with at least one or more of the cells on the other structural member, the open front sides of the cells each having the shape that is defined by the shape surrounded by line segments P-S-Q-S2-R-S1-P, where when an imaginary right-angled isosceles triangle ABC, where A refers to a vortex and BC refer to two equal sides, is given, S refers to any arbitrary point other than points A, R that is located along the line segment A-R connecting between vortex A and midpoint R on the base B-C, P refers to any arbitrary point other than points A, B that is located along the hypotenuse A-B, and Q refers to any arbitrary point other than points A, C that is located along the hypotenuse A-C and where S1 refers to the point where the line segment P-S will intersect with the base B-C when the line segment P-S connecting between points P and S is turned about the point P, and S2 refers to the point where the line segment Q-S will intersect with the base B-C when the line segment Q-S connecting between points Q and S is turned about the point Q

2. The apparatus for mixing and/or crushing substances into fine particles as defined in claim 1, wherein point P refers to a midpoint along the hypotenuse A-B, and point Q refers to a midpoint along the hypotenuse A-C.

3. The apparatus for mixing and/or crushing substances into fine particles as defined in claim 1 or 2, wherein the area surrounded by the line segment P-S-Q-S2-R-S1-P is equal to one half the area of the imaginary right-angled isosceles triangle ABC.

4. The apparatus for mixing and/or crushing substances into fine particles as defined in any one of claims 1 through 3, wherein the fluid flow path is formed in the axial direction of the cylindrical casing or in the direction perpendicular to the axial direction of the cylindrical casing.

5. The apparatus for mixing and/or crushing substances into fine particles as defined in any one of claims 1 through 4, wherein the first structural member and second structural member are mountable inside the cylindrical casing, and wherein the fluid flow path is formed in the axial direction of the cylindrical casing or in the direction perpendicular to the axial direction of the cylindrical casing.

6. The apparatus for mixing and/or crushing substances into fine particles as defined in any one of claims 1 through 4, wherein the first structural member is the cylindrical casing, and wherein the first structural member includes a plurality of first cells each having its front side open, the plurality of first cells being formed on the inner circumferential wall of the cylindrical casing, and the second structural member includes a plurality of second cells each having its front side open, the plurality of second cells being formed on the outer circumferential wall of the structural member that corresponds to the second structural member being mounted inside the cylindrical casing.

7. The apparatus for mixing and/or crushing substances into fine particles as defined in claim 5 or 6, wherein the cylindrical casing has the divisible construction, wherein the second structural member corresponding to the structural member being mounted inside the cylindrical casing can be mounted removably inside the cylindrical casing by dividing the cylindrical casing.

8. The apparatus for mixing and/or crushing substances into fine particles as defined in any one of claims 1 through 4, wherein the first structural member and the second structural member comprise a first plate member having a plurality of cells on one side thereof, each having its front side open, or a second plate member having a plurality of cells on both of the opposite sides thereof, each having its front side open, and wherein the first plate member and the second plate member are mounted inside the cylindrical casing for forming a fluid flow path in the axial direction or in the direction perpendicular to the axial direction of the cylindrical casing.

9. The apparatus for mixing and/or crushing substances into fine particles as defined in claim 8, wherein the plurality of cells on the other side of the second plate member, each having its front side open, are provided on the locations behind the second plate member corresponding to the locations on which the plurality of cells on the one side of the second plate member, each having its front side open, are provided, where the plurality of cells on the other side of the second plate member is turned by a predetermine angle with regard to the plurality of cells on the one side of the second plate member.

10. The apparatus for mixing and/or crushing substances into fine particles as defined in claim 8, wherein the plurality of cells on the other side of the second plate member, each having its front side open, are provided on the locations behind the second plate member that are different from the locations on which the plurality of cells on the one side of the second plate member, each having its front side open, are provided.

11. The apparatus for mixing and/or crushing substances into fine particles as defined in claim 8, wherein the plurality of cells on the other side of the second plate member, each having its front side open, are provided on the locations behind the second plate member that are different from the locations on which the plurality of cells on the one side of the second plate member, each having its front side open, are provided, where the plurality of cells on the other side of the second plate member is turned by a predetermine angle with regard to the plurality of cells on the one side of the second plate member.

12. The apparatus for mixing and/or crushing substances into fine particles as defined in any one of claims 8 through 11, wherein the apparatus further includes a plurality of frame members that are arranged perpendicularly to the axial direction of the cylindrical casing such that they form a plurality of openings arranged like a honeycomb on the upstream side and downstream side of the location where the fluid flow path is formed by the first structural member and second structural member within the cylindrical casing, the plurality of openings providing a communicating path extending in the direction of the cylindrical casing, and any two adjacent frame members are arranged to face opposite each other such that the openings on one frame member are placed in alternate positions with regard to the openings on the other member.

13. The apparatus for mixing and/or crushing substances into fine particles as defined in any one of claims 8 through 11, wherein the cylindrical casing has the divisible construction, and wherein the first structural member comprises of the first plate member and the second structural member comprises of the second plate member can be mounted removably inside the cylindrical casing by dividing the cylindrical casing.

14. The apparatus for mixing and/or crushing substances into fine particles as defined in claim 12, wherein the cylindrical casing has the divisible construction, and wherein the first structural member comprises of the first plate member, the second structural member comprises of the second plate member, and the frame members placed one over the other can be mounted removably inside the cylindrical casing by dividing the cylindrical casing.

15. The apparatus for mixing and/or crushing substances into fine particles as defined in any one of claims 1 through 14, wherein the plurality of first cells on the first structural member, each having its front side open, and the plurality of second cells on the second structural member, each having its front side open, are formed into the same shape.

16. The apparatus for mixing and/or crushing substances into fine particles as defined in any one of claims 1 through 15, wherein the plurality of first cells on the first structural member, each having its front side open, and/or the plurality of second cells on the second structural member, each having its front side open, are arranged like a honeycomb.

17. The apparatus for mixing and/or crushing substances into fine particles as defined in any one of claims 1 through 16, wherein the apparatus further includes an inlet space and outlet space on the upstream and downstream of the location where the fluid flow path is formed by the first structural member and second structural member within the cylindrical casing, and wherein the inlet space has the frustoconical shape whose diameter is increasing from the inlet toward the downstream, and the outlet space has the frustoconical shape whose diameter is decreasing toward the outlet.

18. The apparatus for mixing and/or crushing substances into fine particles as defined in claim 17, wherein a structural member having the frustoconical shape is disposed in each of the inlet space and outlet space for forming a fluid flow path between the inner circumferential wall of the inner space and the outer circumferential wall of the said structural member having the frustoconical shape, and between the inner circumferential wall of the outlet space and the outer circumferential wall of the said structural member having the frustoconical shape, respectively.

19. The apparatus for mixing and/or crushing substances into fine particles as defined in any one of claims 1 through 18, wherein the first structural member and/or the second structural member are and/or is made of any one selected from a group consisting of carbons, composite metal materials including carbons combined with other metals, ceramics, and minerals.

20. The apparatus for mixing and/or crushing substances into fine particles as defined in any of claims 1 through 18, wherein the cylindrical casing is made of any one selected from a group consisting of carbons, composite metal materials including carbons combined with other metals, ceramics, and minerals.

21. The apparatus for mixing and/or crushing substances into fine particles as defined in any of claims 1 through 18, wherein the first structural member and/or the second structural member are and/or is made of any one selected from a group consisting of resins and synthetic resins.

22. The apparatus for mixing and/or crushing substances into fine particles as defined in any of claims 1 through 18, wherein the cylindrical casing is made of any one selected from a group consisting of resins and synthetic resins.

23. The apparatus for mixing and/or crushing substances in the form of a fluid into fine particles as defined in any of claims 1 through 22, further including a magnet that is mounted on the outer circumference of the cylindrical casing.

24. The apparatus for mixing and/or crushing substances into fine particles as defined in any of claims 1 through 23, further including any one or ones of ultrasonic illuminating means, electromagnetic wave illuminating means, high-frequency wave illuminating means and laser beam illuminating means that is or are linked to the upstream and/or downstream side of the apparatus.

25. The apparatus for mixing and/or crushing substances into fine particles as defined in any of claims 1 through 24, further including an inlet that is linked to the upstream and/or downstream side of the apparatus through which different types of substances are fed.

26. A method for mixing and/or crushing substances into fine particles, wherein the method comprises the steps performed by using the apparatus as defined in any one of claims 1 through 25, introducing a fluid, in which the substances to be crushed into fine particles are mixed, under the applied pressure through the inlet of the cylindrical casing in the apparatus, and crushing the substances into fine particles while flowing the fluid through the cylindrical casing down to the outlet of the cylindrical casing.

27. A method for mixing and/or crushing substances into fine particles, wherein the method comprises the steps performed by using the apparatus as defined in any one of claims 1 through 25, introducing a fluid, in which the substances to be crushed into fine particles are mixed, under the applied pressure continuously through the inlet of the cylindrical casing in the apparatus, placing the fluid under the continuous critical or ultracritical conditions, and crushing the substances into fine particles under the continuous critical or ultracritical conditions while flowing the fluid through the cylindrical casing down to the outlet of the cylindrical casing.
